(19) United States
(12) Patent Application Publication
(10) Pub. No.: US 2024/0225731 A1
(43) Pub. Date: Jul. 11, 2024

(54) PHOTO-THERMAL TARGETED TREATMENT AND SAFETY SYSTEM AND ASSOCIATED METHODS FOR EFFICACY, CONSISTENCY, AND PAIN MINIMIZATION

(71) Applicant: Accure Acne, Inc., Boulder, CO (US)

(72) Inventors: Henrik Hofvander, Limhamn (SE); Aubrey Jean Eck, Arvada, CO (US); Michael John Estes, Lafayette, CO (US)

(73) Assignee: Accure Acne, Inc., Boulder, CO (US)

(21) Appl. No.: 18/605,707

(22) Filed: Mar. 14, 2024

Related U.S. Application Data
(63) Continuation-in-part of application No. 17/735,056, filed on May 2, 2022.
Application No. 17/735,056 is a continuation of application No. 16/658,818, filed on Oct. 21, 2019, now Pat. No. 11,317,969.
(60) Provisional application No. 63/451,991, filed on Mar. 14, 2023.
(60) Provisional application No. 62/771,523, filed on Nov. 26, 2018.
(60) Provisional application No. 62/749,104, filed on Oct. 22, 2018.

(51) Int. Cl. A61B 18/20; A61B 17/00; A61B 18/00

(52) U.S. Cl. CPC A61B 18/203; A61B 2017/00199; A61B 2017/00973; A61B 2018/00029; A61B 2018/0047; A61B 2018/00577; A61B 2018/00702; A61B 2018/00791; A61B 2018/00803; A61B 2018/00988

(57) ABSTRACT

A method for determining parameters for operating a light source within a photo-thermal targeted treatment system for targeting a chromophore embedded in a medium includes: 1) administering a laser pulse at a preset power level below known pain and damage thresholds to a location to be treated; 2) measuring a skin surface temperature at the location; 3) correlation fitting a relationship between the light source parameters and the skin surface temperature at the location; 4) defining a safe operating range for the light source parameters to avoid pain and thermal damage at the location; 5) maintaining the skin surface temperature below the known pain and damage threshold and increasing the peak temperature and of the thermal gradient depth; and 6) administering a higher-level laser pulse to raise the temperature of the targeted chromophore to its required damage temperature.

### REFERENCE TO RELATED APPLICATIONS

The present application claims the benefit of U.S. Provisional Application Ser. No. 63/451,991, filed 2023 Mar. 14 and titled “Photo-Thermal Targeted Treatment System and Associated Methods for Efficacy, Consistency, and Pain Minimization.” The present application is a continuation-in-part of co-pending U.S. patent application Ser. No. 17/735,056, filed 2022 May 3 and titled “Determination Process and Predictive Closed-Loop Control of Dosimetry Using Measurement of Skin Surface Temperature and Associated Methods.” The above application is in turn a continuation of U.S. patent application Ser. No. 16/658,818, now U.S. Pat. No. 11,317,969, filed 2019 Oct. 21 and titled “Dosimetry Determination Process via Measurement of Skin Surface Temperature and Associated Methods,” which claims the benefit of U.S. Provisional Patent Application Ser. No. 62/749,104, filed 2018 Oct. 22 and titled “Dosimetry Determination Process via Measurement of Skin Surface Temperature and Associated Methods,” as well as U.S. Provisional patent Application Ser. No. 62/771,523, filed 2018 Nov. 26 and titled “Predictive Closed-Loop Control of Dosimetry Using Measurement of Skin Surface Temperature and Associated Methods.” All of the above-referenced applications are incorporated herein by reference in their entirety.

### FIELD OF THE INVENTION

The present invention relates to photo-thermal targeted treatment systems and, more specifically, systems and methods for controlling the safety operations of a photo-thermal targeted treatment system for improved efficacy, consistency, and pain minimization in providing photo-induced thermal treatment targeting specific chromophores embedded in a medium.

### DESCRIPTION OF RELATED ART

Chromophores embedded in a medium such as the dermis, can be thermally damaged by heating the chromophore with a targeted light source, such as a laser. However, the application of enough thermal energy to damage the chromophore can also be damaging to the surrounding dermis and the overlying epidermis, thus leading to epidermis and dermis damage as well as pain to the subject. This problem also applies to targets, such as sebaceous glands, where a chromophore such as sebum is used to heat the target to a high enough temperature to cause damage to the target.

Previous approaches to prevent epidermis and dermis damage, as well as subject pain include:
1. Pre-cooling the epidermis, then applying the photo-thermal treatment; and
2. Pre-cool the epidermis, also pre-condition (i.e., preheat) the epidermis and dermis in a preheating protocol, then apply photo-thermal treatment in a distinct treatment protocol. In certain instances, the preheating protocol and the treatment protocol are performed by the same laser, although the two protocols involve different laser settings and application protocols, thus leading to further complexity in the treatment protocol and equipment

More recent approaches have involved, for example, determination and predictive closed-loop control of dosimetry using measurement of skin surface temperature, as discussed in the above mentioned related disclosures. Additionally, there has been a shift in such photo-thermal treatment systems to provide more efficient and consistent treatment results while considering patient comfort.

Thus, there is a need for an improved photo-thermal targeted treatment system and methods for providing effective, consistent treatment results and minimizing the pain felt by patients during the course of treatment.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In accordance with the embodiments described herein, there is disclosed a method for determining a suitable set of parameters for operating a light source within a photo-thermal targeted treatment system for targeting a chromophore embedded in a medium. The method includes, prior to administering a treatment protocol to a first subject, 1) administering at least one laser pulse at a preset power level to a first treatment location, where the preset power level is below a known damage threshold. The method also includes 2) measuring a skin surface temperature at the first treatment location, following administration of the at least one laser pulse. The method further includes 3) estimating a relationship between the parameters for operating the light source and the skin surface temperature at the first treatment location, and 4) defining a safe operating range for the parameters for operating the light source in order to avoid thermal damage to the medium at the first treatment location while still effectively targeting the chromophore in administering the treatment protocol.

In an embodiment, steps 1) through 4) are repeated at a second treatment location on the first subject prior to administering the treatment protocol at the second treatment location. In another embodiment, steps 1) through 4) are repeated at the first treatment location on a second subject prior to administering the treatment protocol on the second subject. In still another embodiment, the method further includes 5) storing in a memory of the photo-thermal targeted treatment system the safe operating range for the parameters for operating the light source for the first subject at the first treatment location, and 6) when administering the treatment protocol on the first subject at a later time, taking into consideration the parameters so stored in the memory.

In another embodiment, a photo-thermal targeted treatment system for targeting a chromophore embedded in a medium is disclosed. The system includes a light source configured for providing laser pulses over a range of power levels when operated using a set of parameters, the range of power levels including a known damage threshold for the chromophore and a treatment location. The system also includes a temperature measurement apparatus for measuring a skin surface temperature at the treatment location, and a controller for controlling the light source and the temperature measurement apparatus. The controller is configured for estimating a relationship between the parameters of the light source and the skin surface temperature at the treatment location, defining a safe operating range for the set of parameters of the light source in order to avoid thermal damage to the medium at the treatment location while still effectively targeting the chromophore in administering the treatment protocol, and setting the light source to administer the laser pulses within the safe operating range.

In yet another embodiment, a method for adjusting a suitable set of parameters for operating a light source within a photo-thermal targeted treatment system for targeting a chromophore embedded in a medium during administration of a treatment protocol to a first subject at a first treatment location is disclosed. The method includes: 1) measuring the skin surface temperature at the first treatment location at least once; 2) predicting skin temperature as the treatment protocol is administered to the first subject at the first treatment location; and 3) adjusting at least one of the parameters for operating the light source such that a future measurement of the skin surface temperature at the first treatment location will not exceed a specified value. Predicting skin temperature considers at least one of a heat transfer model and a series of experimental results.

In another embodiment, a method for determining a suitable set of parameters for operating a light source within a photo-thermal targeted treatment system for targeting a chromophore embedded in a medium is disclosed. The method includes:
1) administering at least one laser pulse from the light source at a preset power level to a location to be treated, the preset power level being below a known pain and damage threshold;
2) measuring a skin surface temperature at the location to be treated;
3) correlation fitting a relationship between the parameters for operating the light source and the skin surface temperature at the location to be treated;
4) defining a safe operating range for the parameters for operating the light source in order to avoid pain and thermal damage to the medium at the location to be treated;
5) maintaining the skin surface temperature below the known pain and damage threshold while simultaneously increasing the peak temperature and depth of the thermal gradient until at the correct depth; and
6) administering at least one higher-level laser pulse from the light source above the known pain threshold and below the damage threshold to raise the temperature of the targeted chromophore to its required damage temperature effectively targeting the chromophore in administering the treatment protocol.

In still another embodiment, a method for determining a suitable set of parameters for operating a light source within a photo-thermal targeted treatment system for targeting a chromophore embedded in a medium is disclosed. The method includes, prior to administration of a treatment protocol to a first subject:
1) Cooling a first treatment location, wherein the cooling includes directing an air flow on the first treatment location;
2) administering at least one laser pulse from the light source at a preset power level to the first treatment location on the first subject, the preset power level being below a known pain and damage threshold;
3) measuring a skin surface temperature at the first treatment location, following administration of the at least one laser pulse;
4) estimating a relationship between the parameters for operating the light source, post-pulse cooling and the skin surface temperature at the first treatment location by fitting the skin surface temperature and parameters for operating the light source using data correlations, wherein the predictor parameters are established using computational analysis taking into account a-priori knowledge of the correlation from clinical experiments;
5) defining a safe operating range for the parameters for operating the light source in order to stay below the pain threshold to the medium at the first treatment location while still effectively targeting the chromophore in administering the treatment protocol, wherein the safe operating range corresponds to the skin surface temperature between approximately 28° C. and 34° C.;
6) measuring the skin surface temperature at the first treatment location at least once during the treatment protocol;
7) adjusting the safe operating range for the parameters of the light source at the first treatment location, maintaining the skin surface temperature below the known pain threshold while simultaneously increasing the peak temperature and depth of the thermal gradient until at the correct depth, wherein the estimating, defining, measuring, and adjusting are updated continuously during the treatment; and
8) administering defining at least one higher-level laser pulse from the light source above the known pain threshold and below the damage threshold to raise the temperature of the targeted chromophore to its required damage temperature effectively targeting the chromophore in administering the treatment protocol.

In a further embodiment, a method for treating a subject with a photo-thermal targeted treatment system including a light source for targeting a chromophore embedded in a medium is disclosed. The method includes:
a) cooling a first treatment location of the subject from a first surface temperature to a second surface temperature;
b) administering a laser pulse from the light source to the first treatment location;
c) during application of the laser pulse, tracking skin surface temperatures at the first treatment location at using an infrared camera operating at a refresh rate of 25 Hz to 400 Hz; and
d) terminating the treatment protocol based at least in part on the skin surface temperatures so measured.

In a further embodiment, a photo-thermal targeted treatment system for targeting a chromophore embedded in a medium is disclosed. The system includes a cooling unit for providing cooling at a treatment location, a light source for providing laser pulses at the treatment location, a temperature monitoring unit for monitoring a skin surface temperature at the location, and a controller for receiving the skin surface temperature as monitored by the temperature monitoring unit and accordingly controlling operating parameters of the cooling unit and the light source. In embodiments, the controller is configured for directing the light source to administer at least one laser pulse at a preset power to the treatment location, the preset power level being blow a known pain and damage threshold, directing the temperature monitoring unit to measure a skin surface temperature at the treatment location, correlation fitting a relationship between the operating parameters of the light source and the skin surface temperature so measured, defining a safe operating range for the operating parameters of the light source in order to avoid pain and thermal damage to the medium at the treatment location, modifying the operating parameters of at least one of the cooling unit and the light source to administer at least one higher-level laser pulse from the light source to maintain the skin surface temperature below the known pain and damage threshold while simultaneously increasing a peak temperature and depth of a thermal gradient until the peak temperature and depth of the thermal gradient reaches a desired depth within the medium at the treatment location, and directing the light source to administer at least one treatment laser pulse from the light source with a power above the at least one initial laser pulse to raise a temperature of the targeted chromophore to its required damage temperature.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF DRAWINGS

FIG. 1 illustrates an exemplary photothermal targeting treatment system, in accordance with an embodiment.

FIG. 2 illustrates an exemplary scanner arrangement for use with the photothermal targeting treatment system, in accordance with an embodiment.

FIG. 3 shows a schematic of an exemplary set of light pulses suitable for use as the integrated pre-conditioning/photo-treatment protocol, in accordance with an embodiment.

FIG. 4 shows the measured temperature at the skin surface as a function of time as the treatment photo pulses are applied thereto, in accordance with an embodiment.

FIG. 5 shows a flow chart illustrating an exemplary process for analyzing the measured skin surface temperature, predicting the temperature of the skin when subsequent laser pulses and/or additional cooling are applied, then modifying the treatment protocol accordingly.

FIG. 6 shows the measured skin surface temperature for various applied laser pulse powers at similar treatment areas for two different individuals.

FIG. 7 shows a flow chart illustrating an exemplary process for a closed-loop control of the laser system parameters based on real-time skin surface temperature measurements, in accordance with an embodiment.

FIG. 8 shows the measured skin surface temperature resulting from the application of four pulses to a treatment area that are used as data for predicting the subject's skin temperature rise with subsequent pulse application, as well as the resulting curve fit and actual temperature measurements, in accordance with an embodiment.

FIG. 9 is a graph showing the temperature increase immediately following each one of a sequence of laser pulses as a function of depth, in accordance with an embodiment.

FIG. 10 is a graph showing the thermal gradient (TG) profile just prior to the subsequent successive laser pulse, in accordance with an embodiment.

FIG. 11 is a graph showing the TG profile immediate after the application of each successive laser pulse, in accordance with an embodiment.

FIG. 12 is a graph showing the temperature by depth after the application of each successive laser pulse in a standard 6-pulse protocol, in accordance with an embodiment.

FIG. 13 is a bar graph showing an alternative laser pulsing protocol, in accordance with an embodiment.

FIG. 14 is a graph showing the TG profile immediately after the application of each successive laser pulse based on the alternative laser pulsing protocol, in accordance with an embodiment.

FIG. 15 is a simplified graph showing the temperature profiles for the dermis and skin surface temperatures as a function of time when using a continuous wave (CW) laser for achieving and maintaining the desired thermal gradient profile, in accordance with an embodiment.

FIG. 16 is a simplified graph showing the TG profile corresponding to the CW laser application of FIG. 15, in accordance with an embodiment.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

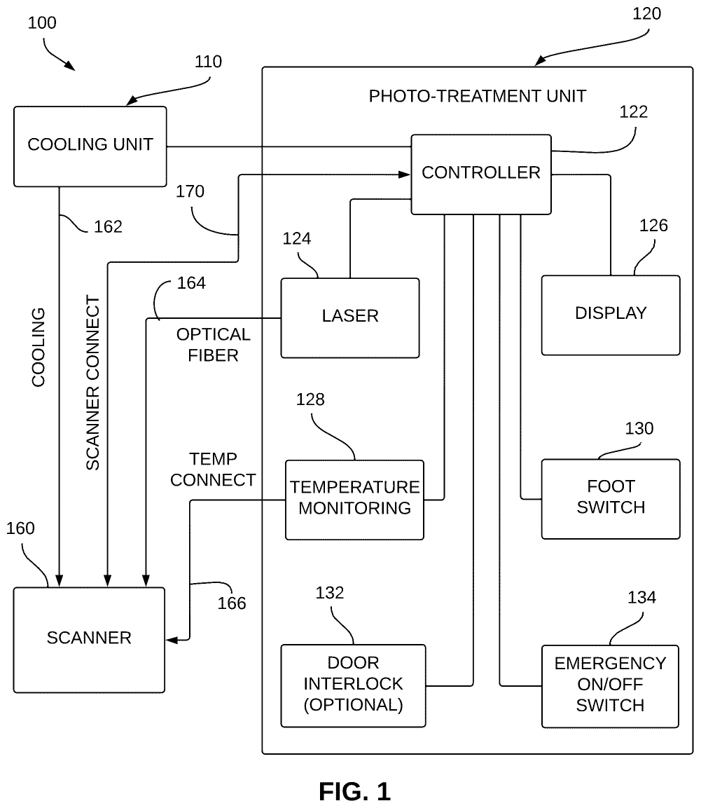

FIG. 1 shows an exemplary photo-thermal targeted treatment system for targeting a target, wherein the target includes specific chromophores embedded in a medium, and heating the target to a sufficiently high temperature so as to damage the target without damaging the surrounding medium. The system can be used, for example, for photo-thermal ablation of sebaceous glands in a targeted fashion, where sebum is the chromophore embedded within the sebaceous gland, while sparing the epidermis and dermis surrounding the target sebaceous glands.

Still referring to FIG. 1, a photo-thermal targeted treatment system 100 includes a cooling unit 110 and a photo-treatment unit 120. Cooling unit 110 provides a cooling mechanism for a cooling effect, such as by contact or by direct air cooling, to treatment area, namely the outer skin layer area overlying the target sebaceous gland. Cooling unit 110 is connected with a controller 122 within photo-treatment unit 120. It is noted that, while controller 122 is shown to be contained within photo-treatment unit 120 in FIG. 1, it is possible for the controller to be located outside of both cooling unit 110 and photo treatment unit 122, or even within cooling unit 110.

Controller 122 further controls other components within photo-treatment unit 120, such as a laser 124, a display 126, a temperature monitoring unit, a foot switch 130, a door interlock 132, and an emergency on/off switch. Laser 124 provides the laser power for the photo-treatment protocol, and controller 122 regulates the specific settings for the laser, such as the output power and pulse time settings. Laser 124 can be a single laser or a combination of two or more lasers. If there more than one laser is used, the laser outputs are combined optically to function as one more powerful laser. Display 126 can include information such as the operating conditions of cooling unit 110, laser 124, and other system status. Temperature monitoring unit 128 is used to monitor the temperature of the skin surface in the treatment area, for example, and the measured skin surface temperature at the treatment area is used by controller 122 to adjust the photo-treatment protocol. Controller 122 also interfaces with footswitch 130 for remotely turning on or off laser 124 and/or cooling unit 110. Additionally, door interlock 132 can be used as an additional safety measure such that, when the door to the treatment room is ajar, door interlock 132 detects the condition and instructs controller 122 to not allow photo-treatment unit 120, or at least laser 124, to operate. Furthermore, emergency on/off switch 134 can be provided to quickly shut down photo-thermal targeted treatment system 100 in case of an emergency. In another modification, additional photodiodes or other sensors can be added to monitor the power level of the energy emitted from laser 124.

Continuing to refer to FIG. 1, photo-thermal targeted treatment system 100 further includes a scanner 160, which is the portion of the device handheld by the user in applying the treatment protocol to the subject. Scanner can be formed, for example, in a gun-like or stick-like shape for ease of handling by the user. Scanner 160 is connected with cooling unit 110 via a cooling connection 162, such that the cooling protocol can be applied using scanner 160. Additionally, the output from laser 124 is connected with scanner 160 via an optical fiber delivery 164, such that the photo-treatment protocol can be applied using scanner 160. Scanner 160 is connected via a temperature connection 166 to temperature monitoring unit 128, so as to feedback the skin temperature at the treatment area, for example, to controller 122. Additionally, the overall operation of scanner 160 as well as feedback from the scanner may be implemented as a scanner connect 170.

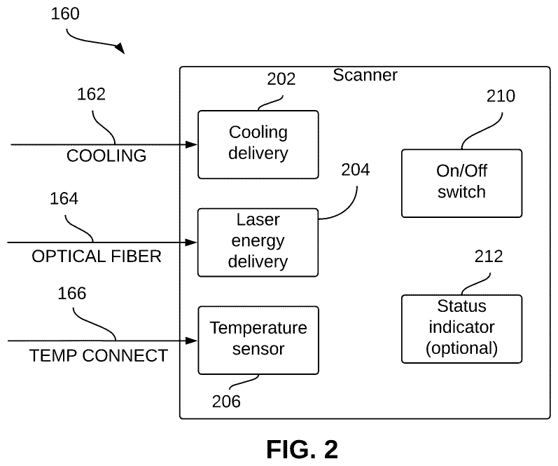

FIG. 2 shows further details of scanner 160, in accordance with an embodiment. Cooling connection 162 is connected with a cooling delivery unit 202, which is configured to deliver the cooling mechanism (e.g., a cold air stream) to the treatment area. Optical fiber delivery 164, from laser 124, is connected with a laser energy delivery unit 204, which includes optical components for delivering light energy for the photo-thermal treatment protocol to the treatment area. For example, the optical components may include a mirror, as controlled by the controller, for steering the light energy in the form of a light beam across multiple treatment spots in an automated manner.

Finally, temperature connection 166 is connected with a temperature sensor 206, which measures the temperature at the treatment area for feedback to controller 122. Additionally, scanner 160 includes an on/off switch 210 (such as a trigger switch to turn on/off laser 124) and, optionally, a status indicator 212, which indicates the operational status of scanner 160, such as if the laser is being operated. While scanner 160 is schematically shown as a box in FIG. 2, the actual shape is configured for ease of use. For example, scanner 160 can be shaped as a nozzle with a handle, a handgun shape, or another suitable shape for ease of aiming and control by the user.

In an exemplary usage scenario, the full treatment area overlying the sebaceous glands to be treated is cooled. The cooling protocol can include, for example, the application of a cold airstream across the treatment area for a prescribed time period, such as 10 seconds. In embodiments, the cooling may be adjusted according to the detection of the skin surface temperature reaching a specified temperature (e.g., −1° C.). Following pre-cooling, the cooling mechanism (e.g., cold airstream or contact-cooling) remains active and a photo-treatment protocol is applied to the treatment area. In one embodiment, pulses of a square, “flat-top” beam are used in combination with a scanner apparatus to sequentially apply a laser pulse to the treatment area. For example, the photo-treatment protocol can include the application of a set number of light pulses onto each segment of the treatment area, with the segments being sequentially illuminated by the laser pulses. In another embodiment, the segments are illuminated in a random order.

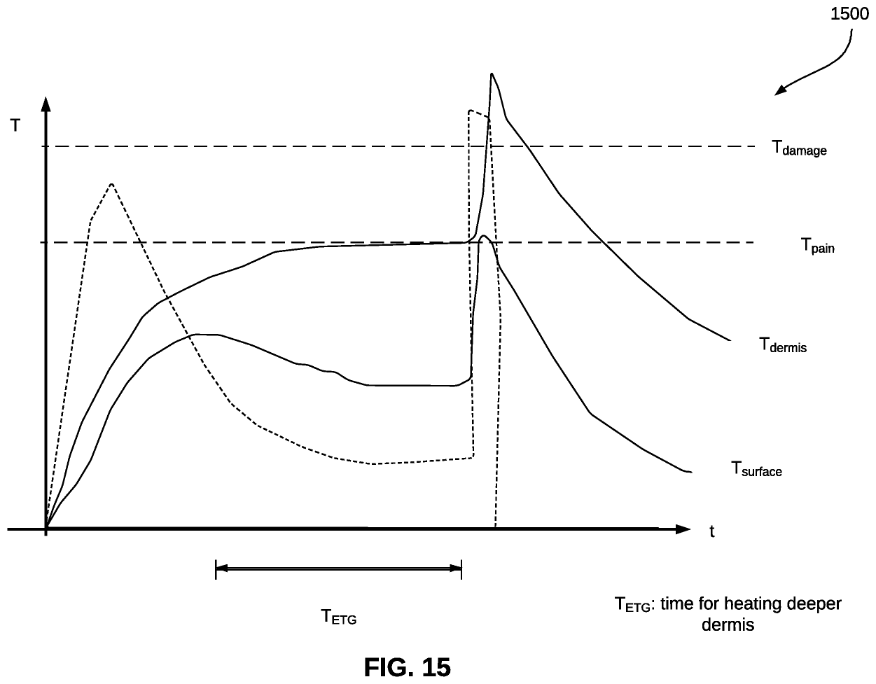

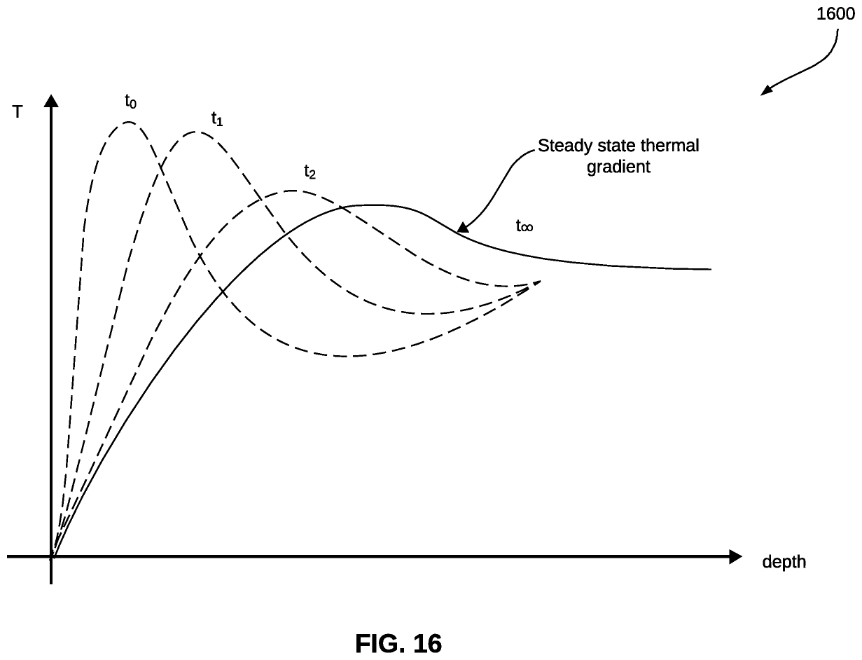

### Power-Modulated CW Illumination (See FIGS. 15 and 16)

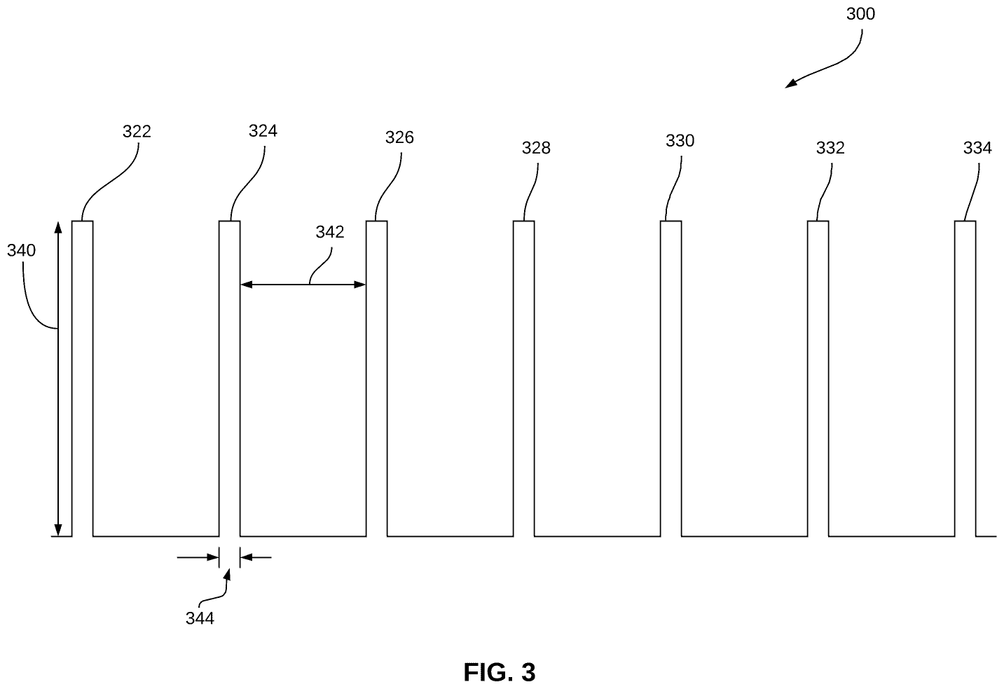

An example of a set of pulses suitable for a conditioning/photo-treatment protocol is illustrated in FIG. 3, in accordance with an embodiment. A sequence 300 includes light pulses 322, 324, 326, 328, 330, 332, and 334 that are applied at the treatment area. In one embodiment, all of the seven light pulses are of equal power and are separated by a uniform pulse separation (represented by a double-headed arrow 342), and have the same pulse duration (represented by a gap 344). In an example, pulse duration 344 is 100 milliseconds, and the pulse separation is 2 seconds. The 2 second pulse separation is intended, for example, to allow the epidermis and dermis in the block to cool down so as to prevent damage thereto. During the pulse separation time period, the laser can be scanned over to a different segment within the treatment area so as to increase the laser use efficiency. It is noted that FIG. 3 is not drawn to scale.

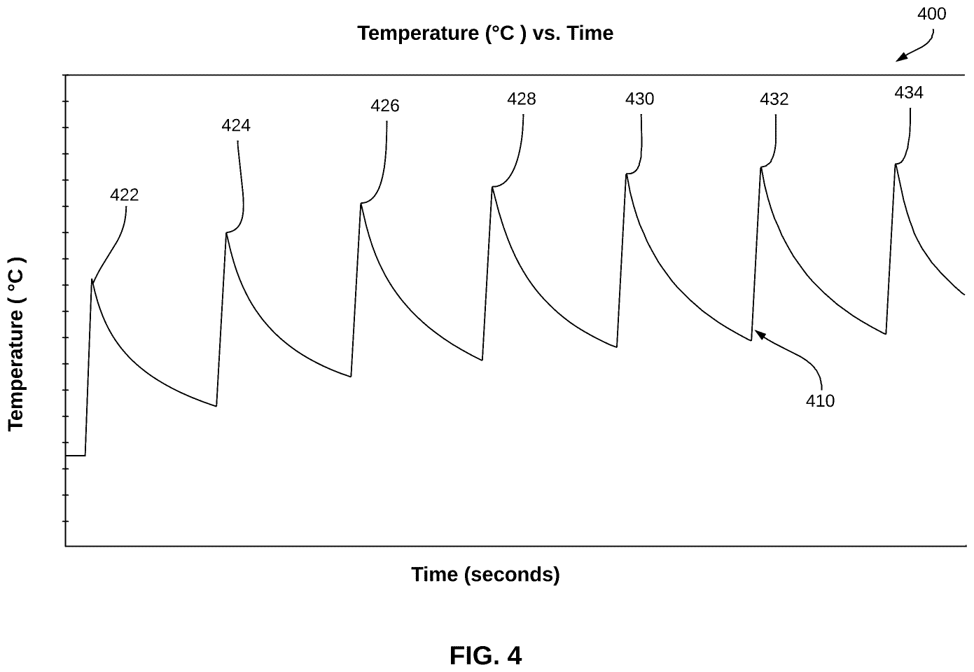

FIG. 4 shows the measured skin surface temperature as light pulses, such as those shown in FIG. 3, are applied to the treatment area, in accordance with an embodiment. In the example shown in FIG. 4, the treatment area had been pre-cooled by direct air cooling for 7 seconds, then light pulses from a 1726 nm wavelength laser at 22 watts power and 100 milliseconds in duration were applied with a period of 2 seconds, while the cooling remains on. In this particular example, the direct-air cooling used for the cooling and during the treatment, delivers a high-speed column of air, cooled to −22° C., resulting in a heat transfer coefficient between the skin and the air of approximately 350 W/m\hat{ }2 K. The beam size is 4.9 mm square, in an embodiment. The exact beam size can be adjusted, using for example collimation optics, depending on the size of the treatment area, power profile of the laser, the location of the treatment area of the body, and other factors.

The resulting changes in skin surface temperature are shown in a graph 400, where a peak 422 corresponds to the application light pulse 322 as shown in FIG. 3, and similarly for peaks 424, 426, 428, and 430. In the example shown in FIG. 4, the average power per spot is 22 W*0.15 s/2 s=1.65 W. The same average power per spot can be achieved, for instance, by pulsing at 33 watts for 100 ms with a pulse separation of 2 s, or by pulsing at 25.1 W for 125 ms with a pulse separation of 1.9 s. Furthermore, the average laser power per area should be in balance with the heat extraction achieved by the cooling system.

The requirements for successful photo-thermal targeted treatment of specific chromophores with minimum subject discomfort include: 1) Epidermis sparing, namely making sure that the peak temperature value at the skin surface is less than around 49° C.; 2) Dermis sparing, namely avoiding overheating the dermis by balancing the peak and average power of the treatment pulses with the heat extraction of the cooling system; and 3) Selective heating of the chromophore, and the target containing the chromophore, such as a peak temperature greater than 55° C. for sebaceous gland treatments. It is noted that the peak temperature value of 55° C. is highly dependent on the specific treatment protocol and is adjustable to other temperatures to stay within a safe operating range to avoid damaging the surrounding dermis and epidermis.

It is known in the literature that tissue parameters, such as the thickness of the epidermis and dermis, vary among individuals, according to factors such as age, gender, and ethnicity, as well as between different skin locations on the body. For example, the forehead has different tissue properties than the back, even for the same individual, thus necessitating different treatment parameter settings for the different treatment locations. Consideration of such variations in tissue properties in determining the specific treatment protocol is significant for laser-based treatment of acne. Additionally, there may be variations in, for instance, the exact laser power, spot size, and cooling capacity between specific laser systems due to manufacturing variability and operating conditions. In fact, manufacturing variations from system to system can result in fluence variation of 15% or more among different laser treatment systems. Furthermore, the individual technique used by the user delivering the treatment can also affect the treatment, e.g., by different pressures applied to the skin surface which in turn affects, for instance, blood perfusion at the treatment site.

In laser treatment of acne, the operating thermal range is generally bound on the upper end at the epidermis and dermis damage threshold temperature, and at the lower end by the temperature required to bring the sebaceous gland to its damage threshold temperature. While there is currently not a good way to directly measure the temperature of the sebaceous gland being targeted by the treatment protocol, the skin surface temperature can be an indicator of the sebaceous gland temperature. A correlation model providing the correspondence between sebaceous gland temperature and skin surface temperature can then be used to tailor the actual treatment protocol using skin surface temperature measurements for effectively targeting sebaceous gland damage while staying below the damage threshold for the epidermis. The correlation model can be developed using, for example, an analytical heat transfer model, or by using clinical data (e.g., via biopsies) correlating skin surface temperature to sebaceous gland damage given the application of a specific treatment protocol.

Based on clinical data, the operating temperature range for acne treatment expressed in terminal skin surface temperature, using for example the treatment protocol illustrated in FIGS. 3 and 4, may be approximately 40° C. to 55° C. When the skin surface temperature is between 45° C. and 55° C. (or even 41° C. to 45° C.), there are varying degrees of sebaceous gland damage, with virtually no epidermal damage. Above 49° C., there is epidermal damage in addition to damage to the sebaceous gland. In examples, staying within a temperature range of 40° C. to 50° C. may be desirable in certain treatment scenarios. In other cases, such as to ensure staying below the pain and damage thresholds of a sensitive patient, it may be desirable to keep the operating temperature range to within 41° C. to 45° C.

However, clinical data also indicates that terminal skin surface temperature has a strong dependence on tissue parameters at the specific treatment area for a particular individual. While existing treatment protocols have been based on a “one treatment fits all” type of an approach, an innovative analysis protocol can be incorporated into the treatment method so as to directly determine individually tailored treatment parameters extrapolating from measurements of terminal skin temperature at lower laser powers and/or the skin surface temperature reached during an initial part of a treatment and/or the terminal skin surface temperatures reached during previous treatments, to avoid epidermis damage while efficaciously causing sebaceous gland damage. In this way, the treatment protocol can be customized for a specific treatment area for a particular individual, and also mitigates treatment variations that can be caused by variations in the laser power output of a specific machine, as well as variations in treatment conditions, such as ambient humidity and temperature. Therefore, it would be desirable to optimize the treatment protocol for different subjects and even different tissue locations for the same subject so as to not cause unwanted tissue damage, while still effectively treating the target tissue component (e.g., the sebaceous gland).

For instance, by directly measuring the skin surface temperature during the first four pulses of FIG. 3, the maximal epidermal surface temperature after application of subsequent pulses can be predicted with a high degree of accuracy. This prediction can be used for real time modification of the specific treatment protocol for a particular area of the skin, such as reducing the number of pulses applied, adjusting the pulse widths, or modifying the laser power, for the subsequent pulses. If the laser system incorporates a cooling system that can react quickly enough, the cooling is also adjustable as part of the real time modification of the treatment system parameters. This customization process greatly enhances subject comfort and safety during the treatment procedure.

This analysis protocol can be performed by incorporating temperature measurements using, for example, a commercial, off-the-shelf, low-cost cameras that can be built into the scanner (e.g., see temperature sensor 206 of FIG. 2) that is held by the medical professional to apply the treatment to the subject, or by using a separate, commercial off-the-shelf single or multi-pixel thermal measurement device. The prediction process can be performed on a highly localized level, thus adjusting the treatment protocol on the fly or prior to the treatment commencement, even adjusting the protocol for each individual spot in a treatment matrix. In this way, the treatment protocol can be specified to provide the necessary treatment laser power while staying below the epidermis and dermis damage threshold temperature.

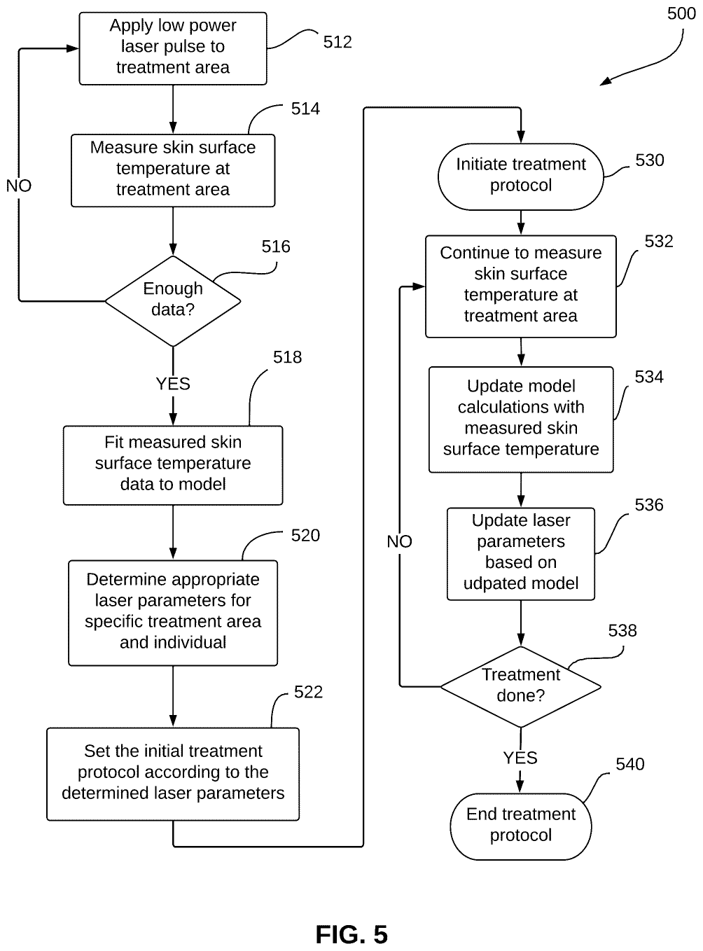

Turning to FIG. 5, a flow chart illustrating an exemplary process for the analysis protocol, in accordance with an embodiment. The analysis protocol assumes that the maximum epidermis temperature and the damage threshold temperature for the target (e.g., the sebaceous gland) are known. Additionally, a correlation model between the skin surface temperature and the target (e.g., the sebaceous gland) has been established using computational analysis, such as finite element modeling of the heat transfer for instance, or by clinical experiments using biopsies. Thus, knowledge of the target value for the terminal skin surface temperature is assumed for the analysis protocol. As an example, for the treatment protocol earlier described in FIGS. 3 and 4, it is known that the target peak skin surface temperature is 51° C.

As shown in FIG. 5, an analysis protocol 500 begins by applying a low power laser pulse to a treatment area in a step 512. The laser power should be set at values that are below the damage threshold for epidermis damage. The skin surface temperature at the treatment area is then measured in a step 514. The temperature measurement can be performed, for example, using a low-speed infrared camera or similar apparatus. Then a determination is made in a decision 516 whether enough data has been collected to fit the collected data into the pre-established correlation model. If the answer to decision 516 is no, then the process returns to step 512, at which a laser pulse at a different, low power setting is applied to the treatment area to gather additional correlation data between applied laser power and epidermis temperature.

If the answer to decision 516 is yes, then analysis protocol 500 proceeds to fit the measured skin surface temperature data to the established correlation model in a step 518. Next, the appropriate laser parameters for the specific treatment area for the particular individual are determined in a step 520. Finally, in a step 522, the exact treatment protocol to be used for the specific treatment area for the particular individual is modified according to the appropriate laser parameters found in step 520.

Continuing to refer to FIG. 5, optionally, analysis protocol 500 can be continued during the actual treatment protocol. In an exemplary embodiment, following the setting of the laser parameters in step 522, a treatment protocol with the appropriate laser parameters is initiated in a step 530. Then, in a step 532, the process continues to measure the skin surface temperature at the treatment area. The measured skin surface temperatures are used to update the correlation model calculations in a step 534, and the laser parameters for the treatment protocol are updated based on the updated calculations in a step 536. Then a decision 538 is made to determine whether the treatment protocol (i.e., the number of laser pulses to be applied to the treatment area) is complete. If the answer to decision 538 is NO, then the analysis protocol returns to step 532 to continue measuring the skin surface temperature. If the answer to decision 538 is YES, then the treatment protocol is terminated in a step 540.

In other words, until the treatment protocol is complete, analysis protocol 500 can implement optional steps 530 through 540 to continue adjusting the laser parameters even during the actual treatment protocol. In fact, if other relevant data regarding the subject, such as laser settings from prior treatments in the same treatment area for the same subject, exist, they can also be fed into the model calculations for further refinement of the laser parameters.

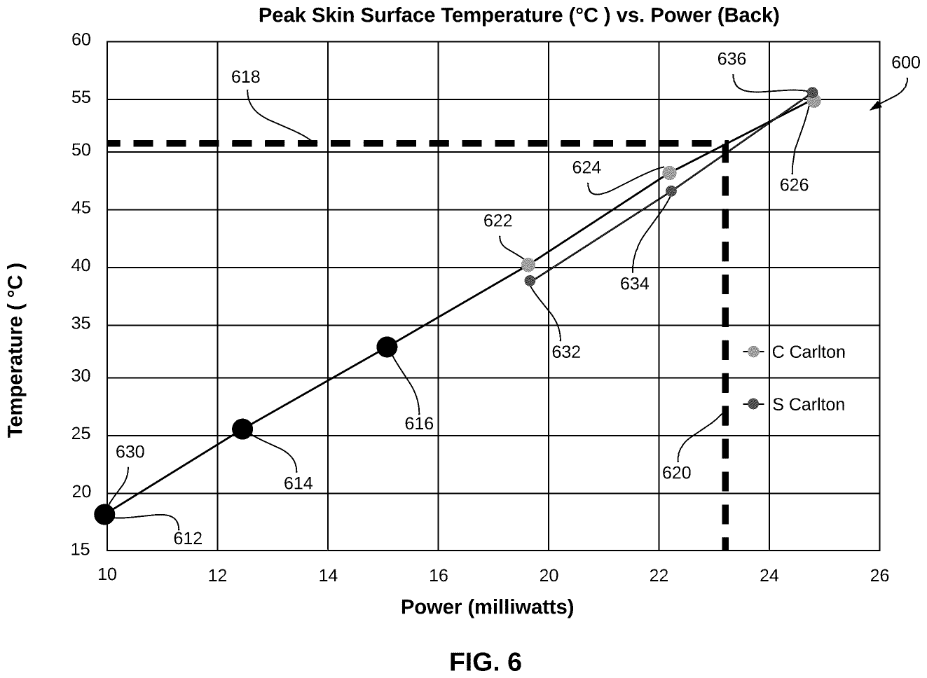

Turning now to FIG. 6, an example of the analysis protocol and subsequent treatment protocol are shown, in accordance with an embodiment. A graph 600 shows the relationship between laser power and peak skin surface temperature during application of a series of laser pulses on two different subjects, identified as “C Carlton” and “S Carlton.” Large dots 612, 614, and 616 show the initial three low power laser pulses applied to subject “C Carlton,” after which the analysis protocol described above is used to predict peak skin surface temperature as measured by an IR camera, thus defining a safe operating range as indicated by a dashed horizontal line 618 and a dashed vertical line 620. Dots 622, 624, and 626 show data take at slightly higher laser power settings on same subject “C Carlton.”

Continuing to refer to FIG. 6, in order to determine the applicability of the same dosimetry determination process on a different subject, the same power laser pulses were applied to a second subject “S Carlton,” starting with a similar starting temperature as shown by a dot 630. On the second subject “S Carlton,” a treatment protocol of increasing laser power was immediately applied, without the dosimetry protocol at lower temperatures, as shown by dots 632, 634, and 636. While the actual measured epidermis temperature for second subject “S Carlton” differed from those of first subject “C Carlton,” graph 600 indicates that the safe operating range, indicated by dashed lines 618 and 620, would have been also applicable for second subject “S Carlton” as well. In this way, the analysis protocol described above takes into consideration these individual differences in tailoring the treatment protocol for a specific treatment area on a particular individual. The efficacy of the analysis protocol has been verified with in vivo data.

The analysis protocol can be performed in advance of the actual treatment session, for example, as the subject is being checked in at an appointment or in a pre-treatment session. As low powers are used, the analysis protocol can be performed without the need for local anesthesia, with virtually no epidermal or dermal damage occurring during the application of the analysis protocol. For instance, in preparation for treatment, a trained operator can quickly pre-measure the various treatment locations and, with one scan per skin location, develop an individualized treatment protocol. Alternatively, the temperature adjustment, including adjustment of laser power and/or cooling mechanism, may be performed in real time, during the actual treatment protocol.

Once a relationship between laser power and the resulting skin surface temperature has been established for a particular subject, and/or a particular skin location, and/or a particular laser device, this relationship, indicated by the slope of the line connecting dots 612, 614, 616, 622, 624, and 626 in FIG. 6, can be used to continuously adjust future treatments. Furthermore, as the treatment schedule proceeds, all of the treatment data can be added to the basis for establishing the skin surface versus power correlation. In this way, the correlation is continually updated and refined, even after the treatment protocol is initiated. For instance, based on the known relationship between laser power and resulting skin surface temperature reached at a particular treatment location, a suggestion for adjusting the laser parameters, such as the laser power can be given to the dermatologist for manual adjustment, or the device can automatically adjust e.g. the laser power, for the next treatment location.

The concept of the analysis protocol described above can be extended to real-time adjustment of the treatment protocol using a closed-loop control process. The surface temperature of the skin can be measured using, for example, an infrared (IR) camera or other temperature measurement mechanisms. By fitting the measured temperature to a mathematical model of the skin tissue, for instance, the measured skin surface temperature can be correlated to the temperature of the target component, such as the sebaceous gland, which cannot be directly measured.

That is, in accordance with another embodiment, a system whereby temperature measurements of a skin surface during initial parts of a treatment for a specific location are used to predict future temperatures of the skin surface at this specific location. The future temperatures, so predicted, are used to adjust a thermal energy delivered by a laser, or lasers, by adjusting one or more parameters, such as laser power, pulse width, number of pulses, and others that affect the thermal energy delivered by the laser, or by adjusting one or more parameters of a cooling system, such as air flow, such that the future temperature of the skin surface for the specific location, and thereby the temperature of the underlying regions and components of the tissue, which cannot be readily measured in a direct manner, reaches a desired value or does not exceed a specified value.

In other words, the dosimetry (e.g., the settings of the light treatment including, for example, power settings for the laser light source) administered to the subject) can be adjusted in real-time by using a predictive control process. For instance, by directly measuring the skin temperature during pulses 322, 324, and 326 shown in FIG. 3, a predicted maximal epidermal surface temperature after application of subsequent pulses can be calculated with a high degree of accuracy. This prediction is accomplished by fitting a mathematical function to the measured epidermal surface temperatures following the application, for example, of three or four treatment pulses. The suitable mathematical function is in turn selected based on knowledge of the pulsing scheme used in the treatment protocol. For example, for the treatment protocol shown in FIG. 3, a variety of curve fitting approaches, such as a single exponential function, may provide an accurate model of the skin surface temperature following subsequent treatment pulse applications. This prediction can then be used in modifying the specific treatment protocol for a particular area of the skin in real time. For instance, the user can modify one or more of the number of additional pulses applied, as well as pulse width and laser power of subsequent pulses. Additionally, if the photo-treatment system includes a sufficiently responsive cooling unit, the cooling applied to the treatment area is also adjustable as a part of the real time modification of treatment system parameters. This customization process greatly enhances patient comfort and safety during the treatment procedure.

The analysis for use in the predictive control process can be performed using a temperature measurement device, for example, a commercial, off-the-shelf, low-cost camera incorporated into the scanner (e.g., temperature sensor 206 of FIG. 2) or by using a separate thermal measuring device, such as a single- or multi-pixel thermal imager. By controlling the size of the target treatment area and specifically measuring the skin surface temperature at the target treatment area, the prediction process can be performed on a highly localized level, thus enabling the medical professional administering the treatment protocol to make adjustments prior to the start of the treatment protocol, in real time during the treatment, or even for each individual spot in a treatment matrix. In this way, the treatment protocol can be administered in a highly customizable way to provide the necessary treatment laser power while staying below the epidermis and dermis damage threshold temperature.

For instance, the Arhenius damage function yields that target damage is exponentially related to peak temperature; subsequently, the peak temperature of the skin surface is correlated to the peak temperature of the target component. In an example, the temperature rise is approximately 180° C./second for irradiation with a 22 W laser over a 5 mm-by-5 mm spot with a 100 ms pulse; in this case, the skin surface measurement should be updated approximately every 2.5 ms, or at 400 Hz, in order for the temperature measurement is to be used as a control input for the treatment protocol. With such a fast temperature measurement method, the laser can be shut off when the measured skin surface temperature reaches a preset threshold value.

Alternatively, a slower temperature measurement device can be used to predict the peak temperature by measuring the temperature rise and fall behavior during the early pulse application in the treatment protocol. The skin surface temperature can be measured during the first few laser pulses applied at the treatment area, and the temperature measurements are used to extrapolate the expected skin surface temperature during subsequent pulse applications such that the energy profile of the subsequent pulses can be adjusted accordingly. For instance, laser parameters such as the laser pulse duration, power, and pulse interval can be adjusted in order to deliver the appropriate amount of energy to the target chromophore while avoiding damage to the surrounding medium.

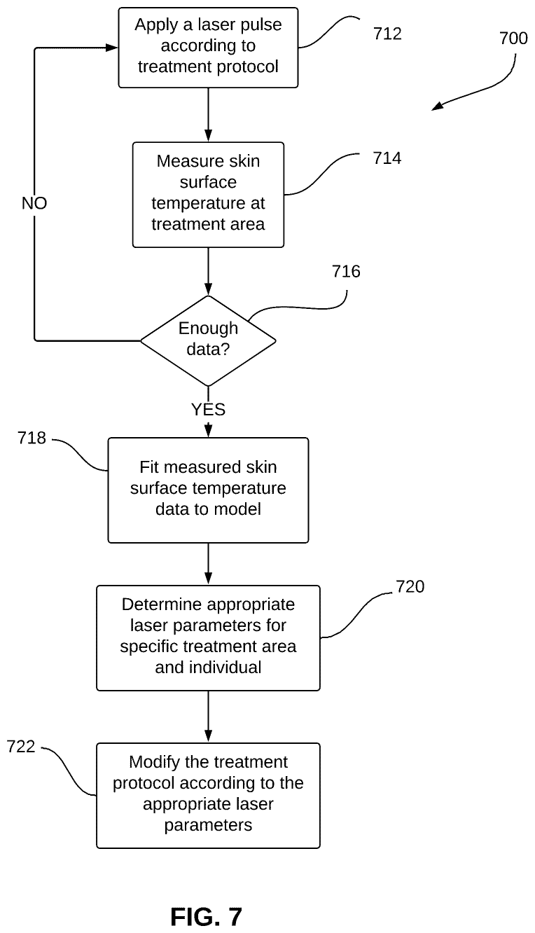

A flow chart shown in FIG. 7 illustrates an exemplary process for a closed-loop control of the laser system parameters based on real-time skin surface temperature measurements, in accordance with an embodiment. A process 700 begins with the initialization of the laser treatment protocol with the laser system set at treatment settings (i.e., therapeutic levels of power, pulse width, etc.). In a step 712, a laser pulse is applied to a treatment area in accordance with a treatment protocol. The treatment protocol can involve, for example, the application of pulses of sequentially increasing power, or pulses of substantially identical power settings repeatedly applied to the treatment area. An example treatment protocol involves the repeated application of laser pulses from a 22 W laser with a 5-millimeter by 5-millimeter spot size and 100 ms duration.

Continuing to refer to FIG. 7, during the application of each laser pulse, the skin surface temperature at the treatment area is measured in a step 714. Optionally, the skin surface temperature is measuring during the cooling down periods between the pulses. The measurement can be made, for example, by a 25 Hz refresh rate infrared camera. Faster devices, such as a 400 Hz refresh rate temperature measurement device, can be used for more accurate measurement of the skin surface temperature at and following the application of the laser pulses.

Then a determination is made in a decision 716 whether enough skin surface temperature data has been gathered for curve-fitting purposes. If the answer to decision 716 is NO, then the process returns to step 712 for the application of another laser pulse. If the answer to decision 716 is YES, then the measured skin surface temperature data are fitted to a predictive model in a step 718. Curve-fits of the maximum and, optionally, minimum skin surface temperatures are generated during step 718. The predictive model can be generated, for instance, by compiling a large number of temperature measurements corresponding to the application of laser pulses to test subjects in clinical settings, or by analytical modeling of the tissue.

Based on the curve-fits generated in step 718, a determination is made in a step 720 for the appropriate laser parameters for the specific treatment area on the individual being treated. In certain embodiments, step 720 may include determining the appropriate laser parameters for the next pulse. For example, if the curve-fits predict the skin surface temperature will rise above a predetermined threshold temperature, such as 45° C., then the laser parameters are adjusted to reduce the laser power. In this case, the skin surface temperature measurements can indicate that the specific treatment area on the subject is particularly sensitive to laser pulse energy absorption. Alternatively, if the curve-fits predict the desired temperature, such as 55° C. for target chromophore damage, will not be reached with the current laser pulse power settings, then the laser parameters can be adjusted to provide the necessary treatment power. Such a situation can occur if the epidermis and dermis characteristics are such that the specific treatment area does not readily absorb the laser pulse energy.

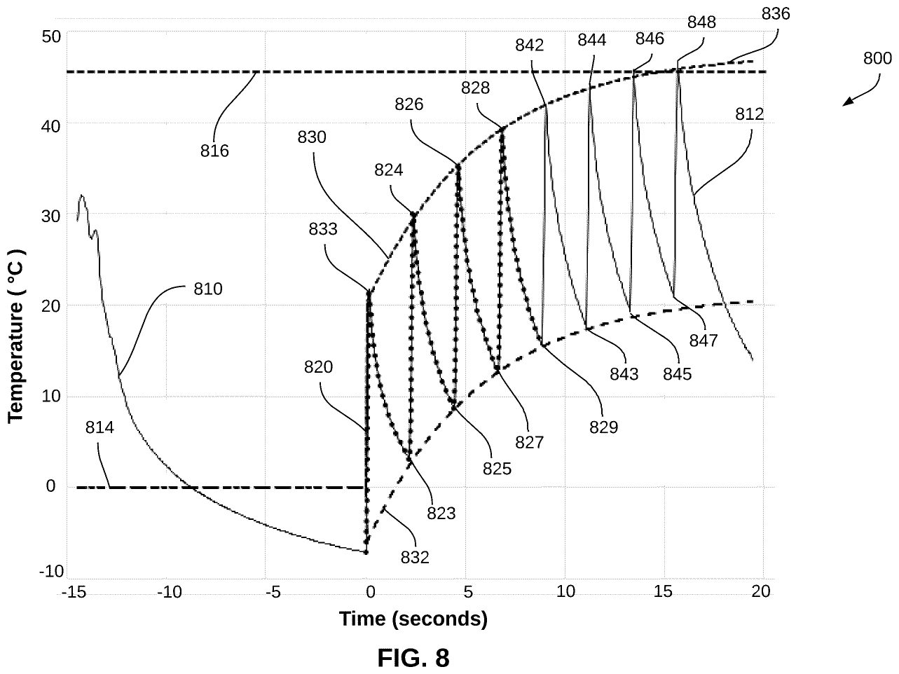

FIG. 8 illustrates an exemplary predictive closed-loop control process based on measured skin surface temperature resulting, in accordance with an embodiment. A graph 800 in FIG. 8 shows the various temperature measurements and calculated curves as a function of time that are used in the predictive closed-loop control process, such as illustrated in FIG. 7. In FIG. 8, time zero corresponds to the moment of application of the first laser pulse (from a 22 W laser, 100 ms pulse and a 5-millimeter by 5-millimeter square spot, in this case) preceded by approximately 15 seconds of air cooling (i.e., time −15 to zero). The air cooling is applied to the treatment area throughout the laser pulse application in the present example. The skin surface temperature is measured using a 25 Hz update rate IR camera in the present example, although the use of other temperature measurement devices is contemplated.

Continuing to refer to FIG. 8, the measured skin surface temperature during the initial cooling is shown by a curve 810. The measured skin surface temperature during the application of laser pulses is shown by curve 812. The target skin surface temperature is indicated by a dashed line 816, shown here at 45.5° C.

Starting at time zero, the first temperature measurements after the first four pulses (indicated by circular dots) are fitted to a predictive model. Specifically, in the example shown in FIG. 8, the peak temperatures as well as the cooled down temperature immediately preceding the next pulse application are fitted into a clinically generated predictive model. Maximum temperature peaks 822, 824, 826, and 828, as well as minimum temperature nadirs 823, 825, 827, and 829, are curve fitted to generate a maximum temperature curve 830 and a minimum temperature curve 832 (shown as dashed curves). Optionally, the temperature measurements made during the cooling down periods between the laser pulse applications are used to improve the measurement accuracy of the maximum temperature peaks as well as the minimum temperature nadirs.

The skin surface temperatures are measured during subsequent laser pulse applications, as shown in curve 812. It can be seen that the maximum and minimum temperature curves 830 and 832 accurately track the measured skin surface temperatures (i.e., peaks 842, 844, 846, and 848 and nadirs 843, 845, and 847 of curve 812). It is noted that the predicted temperature rise (i.e., dashed curve 830) and the actual measured temperatures (specifically peaks 846 and 848) indicate the desired temperature of 45.5° C. has been achieved by the application of pulses 6 and 7, thus the laser treatment protocol is halted without the application of an eighth pulse.

Even with a relatively slow temperature measurement device, such as a 25 Hz refresh IR camera, fitting the temperature data during the cooling down periods between laser pulse applications allows for a good estimation of the rapid temperature rise achieved with each pulse application. If a faster temperature measurement device (e.g., 400 Hz refresh rate or faster) is used, then the temperature profiles can be directly measured in real time.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. For example, lasers with other wavelengths, such as around 1210 nm, can be used. Alternatively, the pre-treatment analysis method described above can be used with other treatment protocols, such as those described in WIPO Patent Application WO/2018/076011 to Sakamoto et al. and WIPO Patent Application WO/2003/017824 to McDaniel. In fact, the method is applicable to any thermal treatment protocol involving equipment that may be subject to system, user, atmospheric conditions, and other variability from treatment to treatment.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

For example, embodiments such as the below are contemplated:
1. A method for determining a suitable set of parameters for a light source within a photo-thermal targeted treatment system for targeting a chromophore embedded in a medium, the method including, prior to administering a treatment protocol: 1) administering at least one laser pulse at a preset power level to a location to be treated, the preset power level being below a known damage threshold; 2) measuring a skin surface temperature at the location to be treated, following administration of the at least one laser pulse; 3) estimating a relationship between the parameters of the light source and the skin surface temperature; and 4) defining a safe operating range for the parameters of the light source in order to avoid thermal damage at the location to be treated.
2. The method of Item 1, wherein steps 1) through 4) are performed on a first subject for a first treatment area, then steps 1) through 4) are repeated on the first subject for a second treatment area.
3. The method of Item 1, wherein steps 1) through 4) are performed on a first subject for a treatment area, then steps 1) through 4) are repeated on a second subject for the treatment area.
4. The method of Item 1, further including taking into consideration treatment data from previous treatments for the same subject.
5. The method of Item 1, wherein steps 2) through 4) are repeated while the actual treatment protocol is performed on the subject.
6. A system whereby temperature measurements of a skin surface during initial parts of a treatment for a specific location are used to predict future temperatures of the skin surface at this specific location. The future temperatures, so predicted, are used to adjust a thermal energy delivered by a laser, or lasers, by adjusting one or more parameters, such as laser power, pulse width, and others that affect the thermal energy delivered by the laser or lasers, or by adjusting one or more parameters of a cooling system, such as air flow, such that the future temperature of the skin surface for the specific location, and thereby the temperature of the underlying regions and components of the tissue, which cannot be readily measured in a direct manner, reaches a desired value.
7. A system whereby temperature measurements of a skin surface taken during a treatment of an adjacent area, or areas, are used to predict future temperatures of the skin surface at this specific location. The future temperatures, so predicted are used to adjust a thermal energy delivered by a laser, or lasers, by adjusting one or more parameters such as laser power, pulse width, and others that affects the thermal energy delivered by the laser or lasers, or by adjusting one or more parameters of the cooling system, such as air flow, such that the future skin surface temperature for the specific location, and thereby the temperature of the underlying regions and components of the tissue, which cannot be readily measured in a direct manner, reaches a desired value.

An improved approach to defining and adjusting the parameters of the photo-thermal treatment system involves the establishment and manipulation of a thermal gradient below the skin surface in the treatment area. Previous discussions of the establishment of a thermal gradient with photo-thermal targeted treatments involve the simultaneous application of a skin surface cooling with a laser pulse to effect damage to a chromophore at a particular depth below the skin surface (see, for example, U.S. Pat. App. Pub. No. 2109-0262072 A1 to Sakamoto, et al., which is incorporated herein by reference). Others have discussed the monitoring of skin surface temperature while applying laser pulses of varying pulse durations and powers in pre-set application protocols, while extrapolating the temperature profile in, for instance, the dermal layer of the treatment area (see, for example, U.S. Pat. No. 8,974,443 B2 to Dunleavy, et al., and U.S. Pat. No. 9,333,371 B2 to Bean, et al.).

In contrast, the embodiments disclosed herein provides systems and methods for controlling the peak of the thermal gradient (TG) established below the skin surface in the treatment area with closed-loop control of the heating and cooling systems. In embodiments, the present disclosure provides for a combination of active cooling and heating, adjustable prior to and during the application of a treatment protocol, to build a desired TG profile below the skin surface. By controlling the TG profile in a closed-loop manner, embodiments of the present disclosure provides consistent management of the photo-thermal energy delivery by the photo-thermal treatment system. In this way, the TG profile at the treatment area may be accurately controlled before and during the treatment, thus providing consistent, efficacious treatment experience for the patient while minimizing the sensation of pain.

In particular, it is recognized herein that the TG profile changes over time at any given location, such that the correlation between a measured surface temperature and the peak TG temperature also changes over time. Thus, it is insufficient to simply monitor the skin surface temperature as an indicator of subcutaneous TG. The relationships between the skin surface temperature, peak TG temperature, TG profile below the skin surface, and time of photo-thermal energy application must be considered to achieve consistent and effective energy delivery while ensuring patient comfort.

The embodiments described herein provide closed-loop control of the TG profile established below the skin surface at the treatment area, including the peak TG temperature during the photo-thermal energy application. As the TG profile at the treatment area changes over time, certain embodiments establish correlations between the skin surface temperature and the peak TG temperature over time, thus enabling active control of the cooling and heating mechanisms of the system to ensure the depth and peak temperature of the TG profile remains below a pain threshold of the patient being treated. In certain embodiments, a pre-treatment TG profile is established at the treatment area prior to any treatment protocol such that, when a treatment pulse is applied at the treatment area, the pain sensation felt by the patient is minimized. For example, the pre-treatment TG profile may be established such that the temperature at the desired target depth below the skin surface is less than 52° C. during the pre-conditioning phase. In embodiments, the method of establishing the TG profile at the treatment area includes using correlation functions to relate the expected heating and/or cooling response to measured variations in the heating and/or cooling response from previously applied photo-thermal energy pulses at a given treatment location.

A key factor differentiating the approaches described in the present disclosure is the recognition that correlation functions are universal for a given heating/cooling protocol, independent of the treatment area location and patient. Prior closed-loop approaches to controlling the photo-thermal energy delivery required adjustments to the cooling and heating parameters between different patients as well as different treatment locations on the same patient. In contrast, the present disclosure describes systems and methods that enable the definition and implementation of correlation functions derived as empirical relationships that allow for accurate predictions of heating and cooling responses across different treatment areas and patients. The correlation functions may be derived from a priori information obtained via laboratory and/or clinical testing, then applied for all subsequent treatments on actual patients.

Particularly, the correlation models implemented in the systems and methods described herein enable the estimation of a temperature drop at a given treatment location as measured between the end of a first heating pulse and just prior to the application of a second heating pulse immediately following the first heating pulse. That is, whereas previous approaches focused on the predictive closed-loop control of the temperature rise measurable at a particular treatment location by application of photo-thermal energy, such as a laser pulse, embodiments of the present disclosure also takes into consideration the cooling drop between the laser pulses to improve the control over the TG established by the laser pulses.

By accurately estimating this cooling drop, the starting temperature at the given treatment location prior to the second heating pulse may be predicted, thus enabling the adjustment of the parameters of this second heating pulse (e.g., power, pulse duration) to ensure that the skin temperature after the application of this second heating pulse may be within an acceptable range of a pre-determined target temperature. This estimation of the cooling drop may be based on the cooling measurements (i.e., the cooling temperature delta) prior to the first laser pulse along with the heating measurements (i.e., the heating temperature delta) of the prior pulse as inputs for the correlation model. That is, the input into the correlation model may also consider any laser pulses applied at the given treatment location prior to the first laser pulse (e.g., any pre-conditioning pulses or prior treatment pulses). In this way, the real time adjustment of the photo-thermal energy delivery based on the correlation model may consider both the cooling and heating temperature deltas at the treatment site, enabling more consistent and accurate control of the photo-thermal energy delivery to the patient than previously possible.

In certain embodiments, the heating properties of the second pulse (e.g., temperature increase per unit power and/or pulse duration) may be estimated from the heating properties any previously applied pulses (e.g., the first pulse and/or any pre-conditioning pulses applied to the given treatment location prior to the second pulse). By incorporating this heating property estimate, the photo-thermal energy source parameters to achieve a target peak temperature may be determined. Additionally, the cooling mechanism may be adjusted during treatment such that the cumulative effects of both the cooling and heating provided at the treatment location may be considered and adjusted as necessary, in real time during the application of the treatment protocol.

In other words, the methods described herein enable the closed loop control of any and all heating pulses (i.e., photo-thermal energy delivery) at a given treatment location. Thus, the described method and systems allow accurate control of the TG and treatment temperatures at different depths. In this way, embodiments of the systems and methods described herein allow more consistent and effective application of photo-thermal energy delivery at a given treatment location than possible using previously disclosed approaches.

As an example, the control of the TG profile by correlation with measured surface temperature may be performed as follows. The TG profile is defined as the profile of temperature as a function of depth in the tissue at a specific point in time. To target specific chromophores at different depths, the TG profile should be controlled to selectively heat those specific chromophores above a damage threshold, while maintaining the surrounding tissue below the damage threshold. Additionally, the pain experienced by the patient during the application of photo-thermal energy may be minimized by maintaining the overall tissue temperature below a predetermined pain threshold for as long as possible.

In order to achieve a desired thermal gradient profile, one may begin with a particular thermal profile in the tissue then apply sufficient photo-thermal energy to provide damage at a desired location within the tissue. Additionally, a cooling mechanism may be provided prior to and/or during the application of the photo-thermal energy. Due to the different mechanisms and efficiency rates of the heating (e.g., application of energy from a pulsed laser) and cooling (e.g., air cooling with the application of cooled air), the heating and cooling must be carefully balanced in order to achieve efficacious treatment results while avoiding tissue damage and patient discomfort. For instance, in order to create a thermal gradient peaked deeper into the tissue, the surface heat extraction and deposition of energy must be balanced near the skin surface while controlling the energy deposition at the desired depth over a sufficient amount of time to achieve the desired effects.

It is also recognized that the TG profile at a given treatment location changes over time. That is, the TG is a function of time, heat extraction rate (i.e., cooling effect produced by any cooling mechanism applied at the tissue surface), heat injection rate (i.e., the tissue heating produced by application of photo-thermal energy at the tissue surface), and tissue depth. Furthermore, for a given heating and cooling protocol, it is recognized that the TG profile as a function of time (i.e., tissue temperature as a function of depth and time) may be estimated using numerical heat transfer modeling using, for example, finite element analysis.

In a generalized three-dimensional case, numerical modeling is generally the only accurate method for estimating the spatial and temporal evolution of the TG profile at a given treatment location. However, the accuracy of such numerical modeling is only as good as the accuracy of the assumed optical and thermal parameters of the tissue used in the simulation. That is, the numerical modeling results may not accurately predict the TG profile in a patient if the actual tissue parameters significantly differ from the assumed optical and thermal parameters used in the simulation. While the numerical simulation results may be validated against clinical and/or experimental results, the simulation results may not accurately predict the TG profile for all patients.

The approaches described in the present disclosure overcomes the shortcomings of the previously described methods by combining the predicted TG profile from photo-thermal heat injection with a refined model of the extraction rate of cooling and real time temperature measurements. In an example, the heat extraction rate of cooling may be described by a known thermodynamic relationship:

$$q = h A Δ T \qquad [ Eq . 1 ]$$

where q is the heat extraction rate (in Watts), h is the heat transfer coefficient (in units of W per m²×x° C.), A is the area being cooled (in m²), and ΔT is the temperature differential between the tissue surface and the cooling medium (in ° C.). Thus, the heat extraction rate may be controlled by changing the heat transfer coefficient (e.g., by changing the cooling air speed, when using air cooling) or the temperature of the cooling medium (e.g., the cooling air temperature, when using air cooling, or the temperature of a contact surface, when using contact cooling).

The depth profile of photo-thermal heat injection may be expressed as a function of the optical absorption and scattering coefficients of the tissue onto which the photo-thermal energy is applied. That is, upon application of photo-thermal energy, the tissue at the applied location will be heated from the surface and into the tissue, where the TG profile depends on the energy and time of the heat application as well as the specific absorption efficient at each tissue layer. In order to provide efficient photo-thermal targeted treatment, it is recognized herein that the damage caused to tissue or a specific structure in the tissue (e.g., the sebaceous gland) generally follows the Arrhenius damage equation with dependence on temperature and time, namely that greater damage may be caused with the application of higher temperatures for a longer time. Therefore, to selectively damage a structure residing at a specific depth without causing damage to the surrounding tissue, the peak temperature of the TG profile should ideally be achieved at the depth of interest where the object to be damaged is located. Further, the sharper the temperature peak, the more selective the intended damage may be provided.

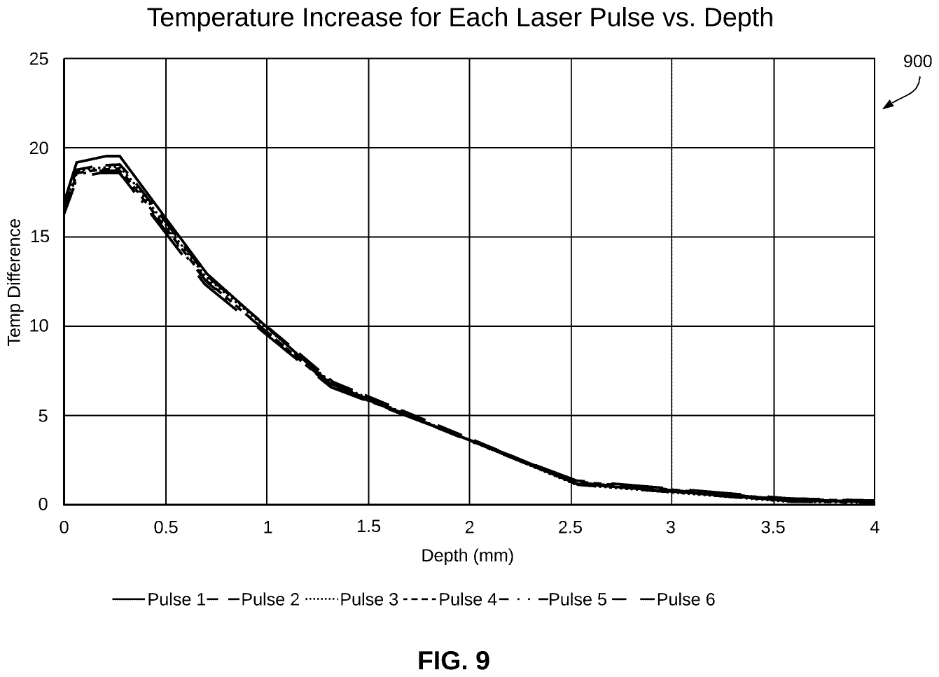

For short optical pulses (e.g., laser pulses with duration less than approximately 0.5 second), the resulting temperature profile of injected heat is generally independent of the temperature of the surrounding tissue if the tissue temperature is above freezing (0° C.) and below the tissue damage temperature (approximately 60-65° C. for pulses less than 0.2 seconds in duration). In such situations, the deposited heat from the short optical pulse has not had time to diffuse into the surrounding tissue prior to the end of the optical heating pulse, and the TG profile of the tissue remains generally unchanged. FIG. 9 illustrates such a situation, showing the temperature difference (i.e., heating temperature delta) immediately after each one of the application of six successive laser pulses to heat the tissue while cooling is simultaneously applied at the same location. In this case, while the heating temperature delta varies with depth of tissue, the temperature profile varies little with the application of the short laser pulses, such as laser pulses of approximately 100 milliseconds or shorter in duration. The curves shown in FIG. 9 are consistent with the common knowledge that photo-thermal sources, such as lasers, microwaves, and incoherent light sources, used for thermal based dermatological procedures tend to most efficiently heat the tissue closer to the source due to the energy absorption by Beer-Lambert Law and optical scattering in the tissue.

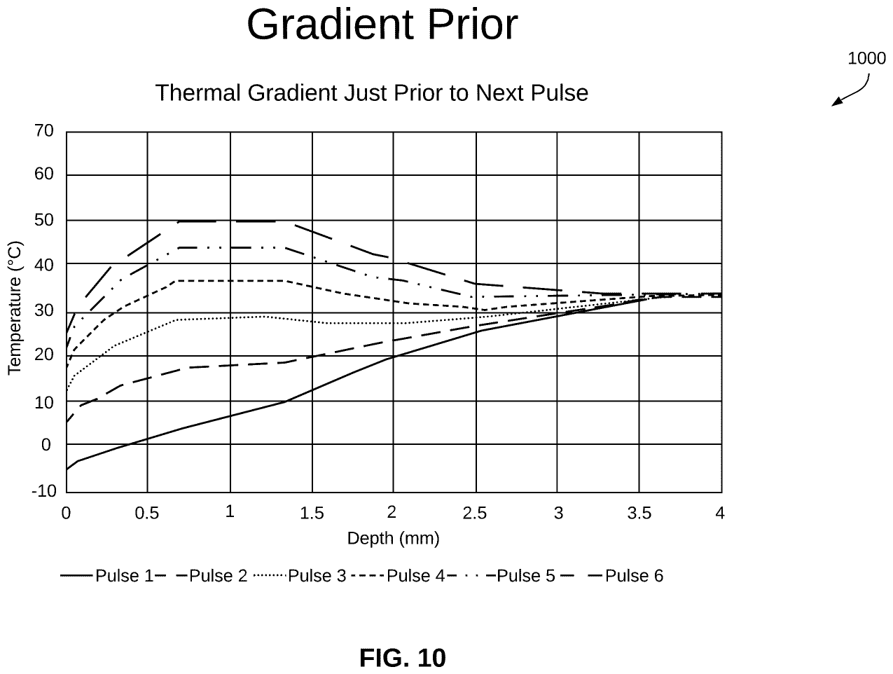

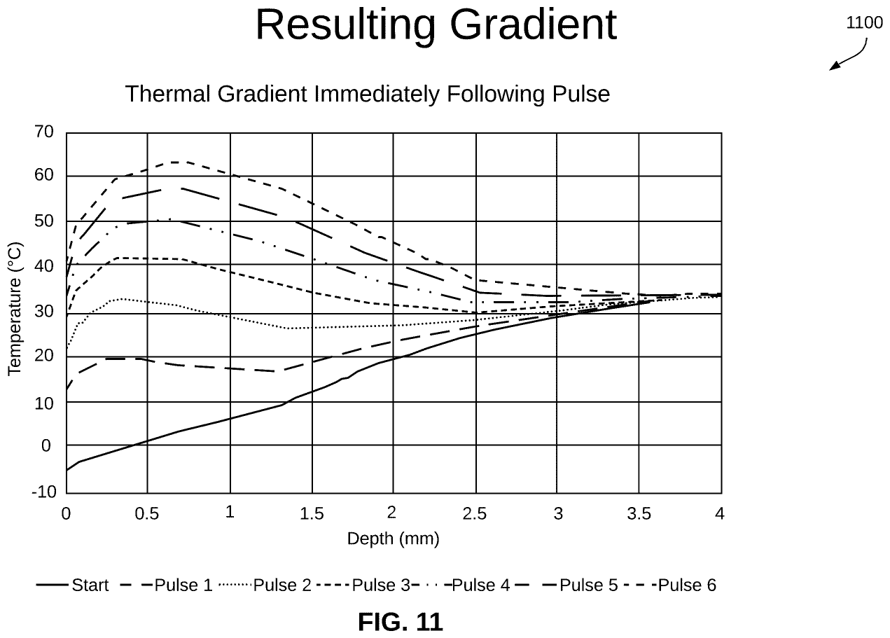

Referring now to FIGS. 10 and 11 in conjunction with FIG. 9, FIG. 10 shows the thermal gradient at a given location, assuming the same six optical pulses used to produce the graph in FIG. 9 are applied to the given location while a cooling mechanism (e.g., air cooling) is applied to the skin surface. FIG. 11 shows the thermal gradient at the same given location immediately following the application of the six pulses of FIG. 9. As shown, the bottom most curves of FIGS. 10 and 11 represent the altered TG profile immediately prior to the first of six optical pulses being applied to the given location. This initial TG profile with only the cooling applied at the skin surface shows that the skin surface temperature is approximately −5° C. and rises toward normal body temperature at increased depth, as expected.

Then, after the first optical pulse has been applied, the second from the bottom curve of FIG. 11 shows the modified TG profile immediately after Pulse 1 of FIG. 9 has been applied to the given location. As can be seen, after the application of Pulse 1, the skin surface temperature (i.e., depth of 0 mm) has increased to above 10° C. with a peak temperature of approximately 20° C. at a dept around 0.4 mm. This TG profile represented by the second from the bottom curve of FIG. 11 settles down to the second from the bottom curve of FIG. 10 immediately prior to the application of Pulse 2. That is, the TG profile represented by the second from the bottom curve of FIG. 10 has been cooled to a skin surface temperature of approximately 6° C. and the temperature peak near 0.4 mm in depth has also been cooled to approximately 14° C. by the time the second pulse is to be applied.

In other words, the TG profiles of the given location continues to fluctuate between a higher peak temperature at a depth of between 0.5 mm and 1 mm immediately after the application of each optical pulse as shown in FIG. 11, then settles down to the slightly flattened curves in FIG. 10. This flattening, corresponding to the cooling temperature delta provided by the continual cooling of the skin surface during the photo-thermal energy application, may lead to errors in the adjustment of the photo-thermal energy parameters if the cooling is not considered.

In previous approaches, as referenced above, the parameter changes were predicated on the peak temperature measurements taken immediately after the optical pulse application, without consideration of the subsequent cooling temperature delta that takes place between the optical pulses. Therefore, while the resulting adjustment of the laser parameters led to providing inconsistent treatment results since the photo-thermal energy provided may not have been sufficient to achieve efficacious treatment results.

However, the embodiments described herein considers both the heating temperature delta provided by the optical pulses as well as the cooling temperature delta provided by the continuous application of cooling at the skin surface. It is recognized herein that the time it takes to cool a given location to a certain depth and a given temperature is a function of the cooling power and the heat diffusion time of the tissue, which is a generally fixed property of the tissue determined by its thermal conductivity, specific heat capacity, and density. While decreasing the temperature of the cooling mechanism and increasing the heat transfer coefficient at the surface of the tissue will speed up the cooling at depths below the skin surface, the upper layers of tissue may freeze with excessive cooling. Therefore, there is a limit to the amount of cooling that may be provided at the skin surface, and it is important to balance the cooling provided and the heating due to the photo-thermal energy application. Taking all of these factors into consideration, the result is a more accurate prediction of the required photo-thermal energy for sufficient heating of the target chromophore and, consequently, more reliable and efficacious photo-thermal targeted treatment results.

For instance, using the thermal model estimations, such as exemplified in the graphs shown in FIGS. 9-11, the TG profiles resulting from the application of photo-thermal energy pulses may be engineered to balance the heat extraction rate (i.e., the cooling power provided by the cooling applied at the skin surface) and cooling time with heat injection and heating time to position the peak temperature of the TG profile at the desired temperature and depth below the skin surface. Since the depth profile of the heat injection for short optical pulses is fixed with a peak temperature at a relatively shallow depth (e.g., approximately 0.3 mm for 1726 laser light, as shown in FIG. 9), the targeting of deeper chromophores (e.g., sebaceous glands located at a depth between approximately 0.5 and 1.5 mm below the skin surface) requires the balancing of the skin surface cooling and time to keep the epidermis and the shallow papillary dermis sufficiently cool to stay below the pain and damage threshold of the patient during application of the laser pulses. With a priori knowledge of the dynamically changing TG profile during photo-thermal energy application, the temperature and depth of the peak temperature of the TG profile may be correlated with the skin surface temperature, thus allowing the peak TG temperature peak to be aimed at the desired location beneath the skin surface.

It is also recognized herein that both excessive heating and excessive cooling may lead to heightened perception of pain by the patient during the pre-conditioning and treatment protocols. Commonly provided pain mitigation procedures include providing sufficient cooling of the skin, the use of a topical anesthetic cream or gel, and the injection of an anesthetic such as those containing lidocaine, the application of a gaseous analgesic such as PRO-NOX™ analgesia, and the like. A combination of the various pain mitigation procedures may also help to reduce the sensation of pain (see, for example, co-pending U.S. patent application Ser. No. 17/564,836 filed Dec. 29, 2021, which is incorporated herein by reference). While the various pain management techniques listed above are effective for most photo-thermal targeted treatment procedures, the injection process of injectable anesthetics is itself painful, various other anesthetics require prescription, and typically requires a physician specialist to perform or supervise the procedures. Thus, it would be beneficial if the pain associated with the photo-thermal targeted therapy itself may be fundamentally reduced.

Specifically taking the example of photo-thermal targeted treatment of acne, the sebaceous glands located 0.5 mm to 1.5 mm below the skin surface are required to be heated to a temperature above 65° C. with a 100 ms laser pulse (for example) to achieve efficacious results. In general, the perception of pain is driven by excess temperature (i.e., temperature above a pain threshold), the time duration of the elevated temperature, and the volume of tissue at these elevated temperature. That is, the pain receptors for elevated temperatures are different depending on the temperature profile. For instance, exceeding the pain threshold at the skin surface will lead to a sensation of heat, whereas exceeding the pain threshold at a depth of approximately 1 mm below the skin surface will lead to a sensation of “needle sticks” at that location. In general, a person will perceive pain based on factors such as: 1) the temperature profile in the tissue, such as the volume of tissue exposed to temperatures above the threshold temperature associated with the pain receptor residing at the applicable depth; and 2) the duration of time spent above the pain threshold. At the depth of the typical sebaceous gland, the pain threshold of an average person is approximately 52° C. (see, for example, Basbaum, et al., “Cellular and Molecular Mechanisms of Pain,” Cell 139, Oct. 16, 2009), and generally no pain is perceived at this depth if the temperature spike is less than 52° C. and the duration is less than 250 milliseconds. As 52° C. is below the damage threshold of the sebaceous gland, additional pain remediation is generally needed with photo-thermal targeted treatment protocols for acne treatment.

As a specific example, consider a photo-thermal treatment protocol targeting a sebaceous gland that resides at a depth of 0.8 mm, without damaging the overlying or underlying dermis. The Arrhenius model shows that sebaceous gland damage is dependent on temperature and time, as is damage to the dermis. Using a time period of 100 milliseconds as an example, the required temperature to damage the target sebaceous gland is 65° C. However, the surrounding dermis must be kept below 60° C. over the same 100 milliseconds.

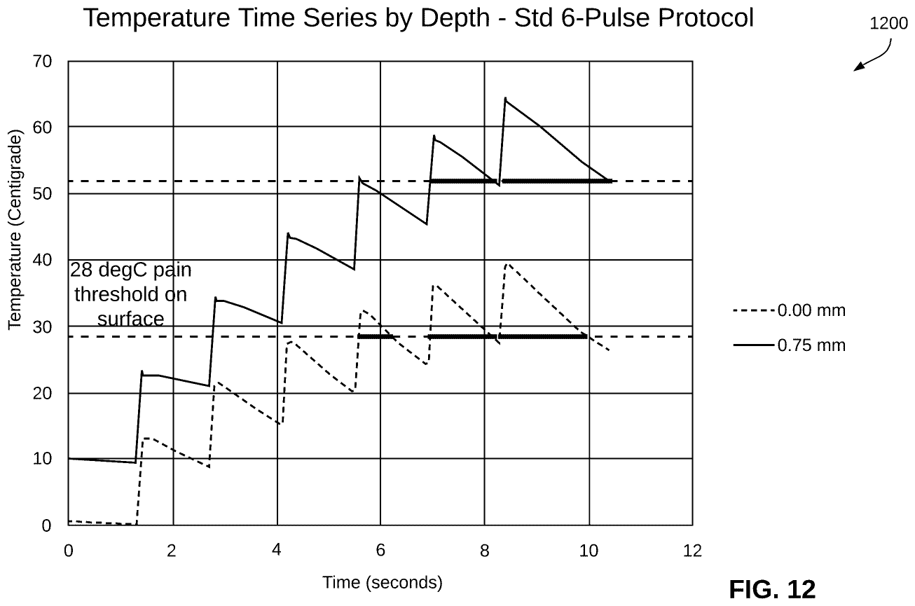

Ideally, the maximum sebaceous gland damage may be achieved without damaging the surrounding tissue by creating a TG profile with a peak temperature above 65° C. at approximately 0.8 mm, taking advantage of the difference in absorption between the sebaceous gland and the surrounding dermis, thus getting the sebaceous gland to reach a temperature above 65° C. while maintaining a dermis temperature below 60° C. Such a TG profile may be achieved by cooling the skin surface using a cooling source with a constant heat transfer coefficient, combined with a photo-thermal energy source such as a laser that deposits energy in a constant fashion, either pulsed or continuous wave. Using a 1726 nm laser, tissue pre-cooling at the skin surface to 1° C. and continuous cooling during the application of laser energy, the peak temperature of the TG profile may be achieved at a depth of 0.8 mm in approximately seven seconds. FIG. 12 illustrates the changes in temperature at depth over a typical six-pulse laser application protocol. However, that time period may be much too long for the patient to tolerate without experiencing excessive pain and/or requiring additional pain mitigation techniques, such as the use of a topical or injected anesthetic.

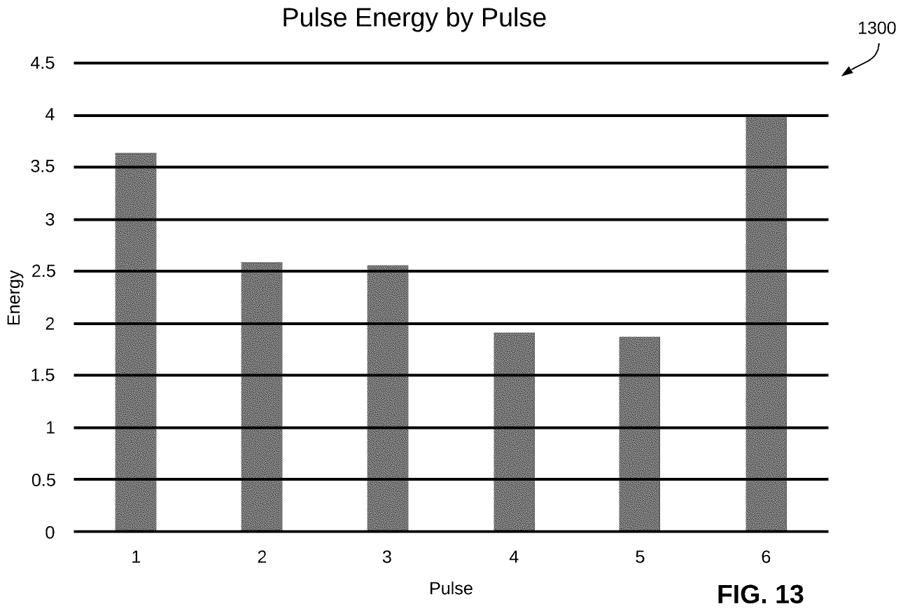

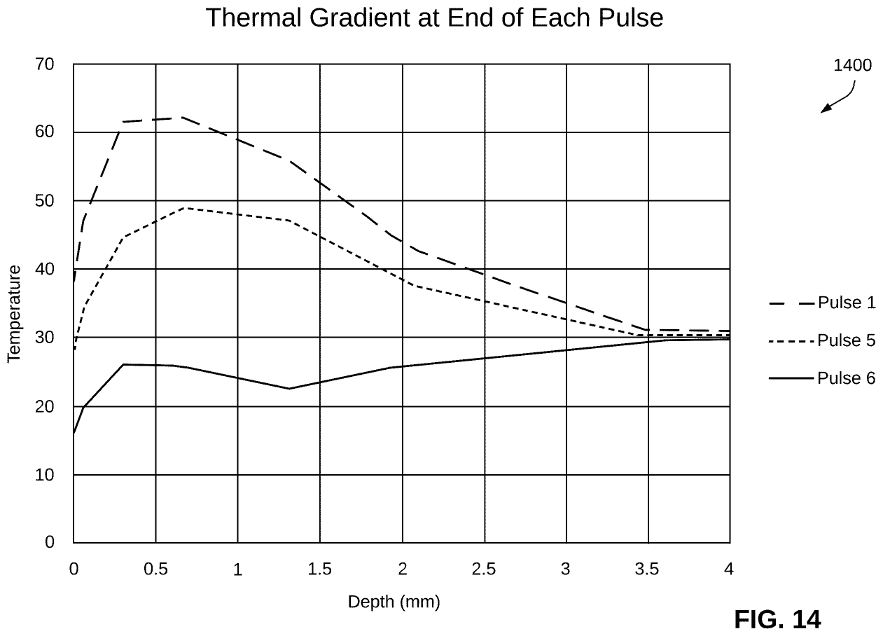

It may be noted upon examination of graph 1200 of FIG. 12 that the temperature at the peak of the thermal gradient beyond 5.5 seconds is above the pain threshold of 52° C. at depth. To avoid this prolonged time period above the pain threshold temperature, embodiments of the present disclosure instead take the approach of first pre-conditioning the initial TG profile, then applying the photo-thermal energy in a way that the peak TG temperature at depth does not exceed the pain threshold until the last pulse. In other words, once the pre-conditioning TG profile has been established at the treatment location, and the peak temperature of the pre-conditioning TG profile is located at the desired depth while being below the pain threshold temperature, a single higher power pulse is delivered to definitively damage the sebaceous gland with a pulse duration shorter than generally perceivable. An example of a pulsing sequence in accordance with an embodiment is shown in FIG. 13. The resulting TG profiles after pulses 1, 5, and 6 of FIG. 13 are shown in FIG. 14.

It is noted that the laser parameters of the pulsing sequence exemplified in FIG. 13 may be specified using the TG profile engineering approach illustrated in FIGS. 9-11. The approach exemplified in FIGS. 13 and 14 minimizes the time spent above the pain threshold of 52° C., thus minimizing the amount of pain experienced by the patient, while still achieving the desired TG profile peak at the targeted depth. In the example curves shown in FIG. 14, it is noted that the peak of the pre-conditioning thermal gradient and the peak of the final thermal gradient are at different depths. Further, it is noted that, while a single high-power pulse is shown in FIG. 13, additional higher-level pulses may be applied, either immediately following the last shot shown in FIG. 13, or by repeating all or a portion of the pulsing sequence shown in FIG. 13.

In certain embodiments, cooling may be provided at the skin surface prior to and/or during the application of the pulses shown in FIG. 13. As a specific example, it is noted that if a fixed cooling mechanism with a fixed heat transfer coefficient is used, care must be taken to avoid reducing the skin surface temperature to below −4° C. for more than approximately six seconds to prevent freezing the skin. Therefore, assuming a seven second laser pulse application sequence, the application of the cooling mechanism at the skin surface may be limited to five seconds of pre-cooling followed by continued cooling during the seven second time period of laser pulse application. In certain situations, the application of the cooling mechanism at the skin surface for additional time may be provided during the pre-cooling period, and the cooling provided at the skin surface may be adjusted during application (e.g., by adjusting the cooling air flow rate) in real time.

In an alternative approach, the combination of the cooling and photo-thermal heating pulses may be adjusted to quickly achieve the peak of the thermal gradient to just below the 52° C. pain threshold at the desired depth early in the seven second laser application period, then reduce the heating (e.g., by reducing the power and/or pulse duration of subsequent pulses) to maintain the peak temperature at the desired depth while allowing time for heat to diffuse, thus heating deeper tissue. In still another approach, the pre-cooling and treatment time may be further reduced by increasing the cooling power by decreasing the temperature of the cooling mechanism or increasing the heat transfer coefficient (e.g., increase cooling airspeed in an air-cooled system). If the skin surface temperate can be maintained just above the freezing temperature during the application of the photo-thermal energy, the additional cooling may more quickly reduce the temperature at depth, thus pushing the peak of the TG profile deeper below the skin surface. Such an effect would allow the application of greater heating power (e.g., increased laser pulse power or duration), effectively resulting in deeper penetration of the heating power into the tissue in a given pulse application sequency while preventing damage at the shallower tissue.

In embodiments, the aim of the processes described herein is to keep the dermis temperature below the generally accepted pain threshold temperature of 52° C. The skin surface temperature and its correlation to the dermis temperature may be determined using the processes described herein and used as a proxy for the temperature at a depth below the skin surface (e.g., at the dermis).

FIG. 15 is a simplified graph showing the temperature profiles for the dermis and skin surface temperatures as a function of time when using a continuous wave (CW) light source for achieving and maintaining the desired thermal gradient profile, in accordance with an embodiment. That is, rather than using laser pulses in the establishment of the initial thermal gradient, a CW light source may be used to establish and maintain the initial thermal gradient profile.

As shown in FIG. 15, a graph 1500 shows the temperature relationships between applied CW power, dermis temperature T_{dermis}, and skin surface temperature T_{surface} as a function of time and relative to the pain threshold Tₚₐᵢₙ and target chromophore damage threshold T_{damage}. Tₚₐᵢₙ and T_{damage} are indicated as dashed horizontal lines.

A CW laser source may be applied at the treatment location, as indicated by a dashed curve in FIG. 15. Consequently, T_{dermis} and T_{dermis} are also elevated over time. In an embodiment, the CW power may initially be rapidly increased to above Tₚₐᵢₙ, then gradually decreased over time. The increase of the CW power to above the pain threshold may be mitigated by the appropriate pre-cooling and/or continued application of cooling to the skin surface, in certain embodiments.

As the CW power is decreased, the wavelength of the CW light source may be selected to promote heating of the deeper dermis while keeping the skin surface temperature at a tolerable level for the subject. This time period is indicated as t_{ETG} in FIG. 15. Then, the laser power may be pulsed to target the chromophore, using the same CW light source or by using a separate pulse laser, to initiate the treatment protocol. The establishment of the thermal gradient using the CW power allows the delivery of sufficient power to raise the target chromophore temperature to above the damage threshold T_{damage} while keeping the skin surface temperature below the pain threshold Tₚₐᵢₙ.

In embodiments, rather than an initial spike of the CW laser power as shown in FIG. 15, the CW power may be gradually increased from a low power to a higher power to establish the thermal gradient. Suitable CW light sources may include, for example, infrared CW lasers, light emitting diodes, and others. Other CW power application schemes may be contemplated and are considered a part of the present disclosure.

FIG. 16 is a simplified graph showing the TG profile corresponding to the CW laser application of FIG. 15, in accordance with an embodiment. As shown in FIG. 16, the depth of the peak temperature beneath the skin surface is shown over time, during the application of the CW power.

At a time to, when the peak CW power is applied as shown in FIG. 15, the peak temperature is closer to the skin surface. As CW power is gradually decreased over time (e.g., at t₁ and t₂), the peak temperature beneath the skin surface gradually moves deeper toward a steady state thermal gradient at a time t_{∞}.

The use of CW power may be advantageous as the steady state temperature at the depth of the target chromophore may be maintained at a higher temperature than with a pulsed pre-heating scheme. Further, the CW power may be applied over a larger surface area at and around the target treatment location, thus enabling the broader heating and earlier establishment of the thermal gradient over a larger surface area.

In addition to the embodiments enumerated above, additional embodiments such as below are contemplated:
1. A method for determining a suitable set of parameters for operating a light source within a photo-thermal targeted treatment system for targeting a chromophore embedded in a medium, the method including:
   1) administering at least one laser pulse from the light source at a preset power level to a location to be treated, the preset power level being below a known pain and damage threshold;
   2) measuring a skin surface temperature at the location to be treated;
   3) correlation fitting a relationship between the parameters for operating the light source and the skin surface temperature at the location to be treated;
   4) defining a safe operating range for the parameters for operating the light source in order to avoid pain and thermal damage to the medium at the location to be treated;
   5) maintaining the skin surface temperature below the known pain and damage threshold while simultaneously increasing the peak temperature and depth of the thermal gradient until at the correct depth; and
   6) administering at least one higher-level laser pulse from the light source above the known pain threshold and below the damage threshold to raise the temperature of the targeted chromophore to its required damage temperature effectively targeting the chromophore in administering the treatment protocol.
2. A method for determining a suitable set of parameters for operating a light source within a photo-thermal targeted treatment system for targeting a chromophore embedded in a medium, the method including, prior to administration of a treatment protocol to a first subject:
   1) Cooling a first treatment location, wherein the cooling includes directing an air flow on the first treatment location;
   2) administering at least one laser pulse from the light source at a preset power level to the first treatment location on the first subject, the preset power level being below a known pain and damage threshold;
   3) measuring a skin surface temperature at the first treatment location, following administration of the at least one laser pulse;
   4) estimating a relationship between the parameters for operating the light source, post-pulse cooling and the skin surface temperature at the first treatment location by fitting the skin surface temperature and parameters for operating the light source using data correlations, wherein the predictor parameters are established using computational analysis taking into account a-priori knowledge of the correlation from clinical experiments;
   5) defining a safe operating range for the parameters for operating the light source in order to stay below the pain threshold to the medium at the first treatment location while still effectively targeting the chromophore in administering the treatment protocol, wherein the safe operating range corresponds to the skin surface temperature between approximately 28° C. and 34° C.;
   6) measuring the skin surface temperature at the first treatment location at least once during the treatment protocol;
   7) adjusting the safe operating range for the parameters of the light source at the first treatment location, maintaining the skin surface temperature below the known pain threshold while simultaneously increasing the peak temperature and depth of the thermal gradient until at the correct depth, wherein the estimating, defining, measuring, and adjusting are updated continuously during the treatment; and
   8) administering defining at least one higher-level laser pulse from the light source above the known pain threshold and below the damage threshold to raise the temperature of the targeted chromophore to its required damage temperature effectively targeting the chromophore in administering the treatment protocol. For example, the laser pulse may provide a temperature elevation above the chromophore damage threshold while remaining below the damage threshold of the surrounding tissue.
3. The method of item 2, further including: repeating steps 1) through 8) at a second treatment location on the first subject prior to administering the treatment protocol at the second treatment location.
4. The method of item 2, further including: repeating steps 1) through 8) at the first treatment location on a second subject prior to administering the treatment protocol on the second subject.
5. The method of item 2, further including:
   9) storing in a memory of the photo-thermal targeted treatment system the safe operating range for the parameters for operating the light source for the first subject at the first treatment location; and
   10) when administering the treatment protocol on the first subject at a later time, taking into consideration the parameters so stored in the memory.
6. The method of item 2, further including:
   9) if the skin surface temperature at the first treatment location reaches a preset threshold temperature, adjusting the parameters of the light source to reduce an effective power incident at the first treatment location.
7. The method of item 2, wherein defining the safe operating range for the parameters of the light source includes setting at least one of laser power, pulse width, pulse interval, maximum power output, and a skin surface cooling mechanism.
8. The method of item 2, further including: repeating steps 1) through 8) at a second treatment location on the first subject during administration of the treatment protocol at the second treatment location.
9. The method of item 2, further including: repeating steps 1) through 8) at a first treatment location on a second subject during administration of the treatment protocol on the second subject.
10. The method of item 9, further including:
   9) storing in a memory of the parameters for operating the light source for the first subject at the first treatment location for the second subject; and
   10) when administering the treatment protocol on the second subject at a later time, taking into consideration the parameters for operating the light source so stored in the memory.
11. A method for determining a suitable set of parameters for operating a light source within a photo-thermal targeted treatment system for targeting a chromophore embedded in a medium, the method including:
   a) cooling a first treatment location;
   b) administering at least one laser pulse from the light source at a preset power level to the first treatment location, the first laser pulse having thermal energy below a known pain and damage threshold of the medium;
   c) tracking skin surface temperatures at the first treatment location at a refresh rate of 25 Hz to 400 Hz while administering the first laser pulse;
   d) estimating a relationship between the parameters for operating the light source, post-pulse cooling and the skin surface temperature at the first treatment location by fitting the skin surface temperature and parameters for operating the light source using data correlations, wherein the predictor parameters are established using computational analysis taking into account a-priori knowledge of the correlation from clinical experiments;
   e) defining a “safe” operating range (i.e., as pertaining to pain) for the parameters for operating the light source in order to stay below the pain threshold to the medium at the first treatment location while still effectively targeting the chromophore in administering the treatment protocol, wherein the safe operating range corresponds to the skin surface temperature between approximately 28° C. and 34° C.;
   f) continuing to track skin surface temperatures at the first treatment location at a refresh rate of 25 Hz to 400 Hz while administering additional laser pulses;
   g) adjusting the safe operating range for the parameters of the light source at the first treatment location, maintaining the skin surface temperature below the known pain threshold while simultaneously increasing the peak temperature and depth of the thermal gradient until at the correct depth, wherein the estimating, defining, measuring, and adjusting are updated continuously during the treatment; and
   h) defining at least one higher-level laser pulse from the light source above the known pain threshold and below the damage threshold to raise the temperature of the targeted chromophore to its required damage temperature effectively targeting the chromophore.
12. The method of item 11, further including repeating steps a.-h. at a second treatment location on the first subject.
13. The method of item 11, further including repeating steps a)-h) on a second subject.
14. The method of item 11, wherein adjusting the parameters for operating the light source includes adjusting at least one of laser power, pulse width, pulse interval, maximum power output, and a skin surface cooling mechanism used for performing the cooling.
15. A method for treating a subject with a photo-thermal targeted treatment system including a light source for targeting a chromophore embedded in a medium, the method including:
   a) cooling a first treatment location of the subject from a first surface temperature to a second surface temperature;
   b) administering a laser pulse from the light source to the first treatment location;
   c) during application of the laser pulse, tracking skin surface temperatures at the first treatment location at using an infrared camera operating at a refresh rate of 25 Hz to 400 Hz; and
   d) terminating the treatment protocol based at least in part on the skin surface temperatures so measured.
16. The method of item 15, wherein cooling the first treatment location includes cooling the first treatment location from the first surface temperature of body temperature to a second surface temperature less than body temperature.
17. The method of item 15, wherein cooling includes using a contact cooling mechanism. In embodiments, the cooling may include cooling by a cooling air flow. In other embodiments, other cooling mechanisms, such as cryogen spray cooling, may be used.
18. The method of item 15, wherein tracking skin surface temperatures includes determining the skin surface temperatures at a refresh rate of at least 400 Hz.
19. The method of item 15, wherein terminating the treatment protocol includes fitting the skin surface temperatures so tracked to data correlations, wherein the predictor parameters are established using computational analysis taking into account a-priori knowledge of the correlation from clinical experiments, determining appropriate laser parameters for the light source, and modifying the treatment protocol according to the appropriate laser parameters.
20. The method of item 19, wherein terminating the treatment protocol further includes determining appropriate cooling parameters for the cooling mechanism, and modifying the cooling parameters during the treatment protocol
21. The method of item 17, wherein terminating a treatment protocol includes fitting the skin surface temperatures so tracked to data correlations, wherein the predictor parameters are established using computational analysis taking into account a-priori knowledge of the correlation from clinical experiments, predicting a peak skin surface temperature, and adjusting at least one of laser power, pulse width, number of pulses, and cooling system parameters.
22. The method of item 21, wherein the peak skin surface temperature is a temperature in a range of 40° C. and 55° C. In embodiments, the peak skin surface temperature may be kept between 41° C. and 45° C. so as to stay below the pain threshold of a wide population of patients.
23. The method of item 22, wherein the peak skin surface temperature is approximately 51° C.
24. The method of item 15, wherein a pulse duration of the laser pulse is 100 milliseconds.
25. The method of item 15, wherein terminating the treatment protocol includes fitting the skin surface temperatures so tracked to data correlations, wherein the predictor parameters are established using computational analysis taking into account a-priori knowledge of the correlation from clinical experiments, determining appropriate laser parameters for the light source, and modifying the treatment protocol according to the appropriate laser parameters.
26. The method of item 25, wherein terminating the treatment protocol further includes determining appropriate cooling parameters for the cooling mechanism, and modifying the cooling parameters during the treatment protocol
27. The method of item 26, wherein terminating a treatment protocol includes fitting the skin surface temperatures so tracked to data correlations, wherein the predictor parameters are established using computational analysis taking into account a-priori knowledge of the correlation from clinical experiments, predicting a peak skin surface temperature, and adjusting at least one of laser power, pulse width, number of pulses, and cooling system parameters.
28. The method of item 27, wherein the peak skin surface temperature is a temperature in a range of 40° C. and 55° C.
29. The method of item 28, wherein the peak skin surface temperature is in a range of 41° C. and 45° C.
30. The method of item 15, wherein a pulse duration of the laser pulse is 100 milliseconds.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”—whether explicitly discussed or not—and, conversely, were there only disclosure of the act of “protruding”, such a disclosure should be understood to encompass disclosure of a “protrusion”. Such changes and alternative terms are to be understood to be explicitly included in the description.

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Therefore, it is to be understood that the foregoing is illustrative of the present invention and is not to be construed as limited to the specific embodiments disclosed, and that modifications to the disclosed embodiments, as well as other embodiments, are intended to be included within the scope of the present disclosure.

1. A method for determining a suitable set of parameters for operating a light source within a photo-thermal targeted treatment system for targeting a chromophore embedded in a medium, the method comprising:
1) administering at least one initial laser pulse from the light source at a preset power level to a location to be treated, the preset power level being below a known pain and damage threshold;
2) measuring a skin surface temperature at the location to be treated;
3) correlation fitting a relationship between the parameters for operating the light source and the skin surface temperature at the location to be treated;
4) defining a safe operating range for the parameters for operating the light source in order to avoid pain and thermal damage to the medium at the location to be treated;
5) maintaining the skin surface temperature below the known pain and damage threshold while simultaneously increasing a peak temperature and depth of a thermal gradient until the peak temperature and depth of the thermal gradient reaches a desired depth within the medium at the location to be treated; and
6) administering at least one treatment laser pulse from the light source with a power above the at least one initial laser pulse to raise a temperature of the targeted chromophore to its required damage temperature to effectively target the chromophore in administering a treatment protocol.

2. A method for determining a suitable set of parameters for operating a light source within a photo-thermal targeted treatment system for targeting a chromophore embedded in a medium, the method comprising, prior to administration of a treatment protocol to a first subject:
1) Cooling a first treatment location, wherein the cooling includes directing an air flow on the first treatment location;
2) administering at least one laser pulse from the light source at a preset power level to the first treatment location on the first subject, the preset power level being below a known pain and damage threshold;
3) measuring a skin surface temperature at the first treatment location, following administration of the at least one laser pulse;
4) estimating a relationship between the parameters for operating the light source, post-pulse cooling and the skin surface temperature at the first treatment location by fitting the skin surface temperature and parameters for operating the light source using data correlations, wherein the predictor parameters are established using computational analysis taking into account a-priori knowledge of the correlation from clinical experiments;
5) defining a safe operating range for the parameters for operating the light source in order to stay below the pain threshold to the medium at the first treatment location while still effectively targeting the chromophore in administering the treatment protocol, wherein the safe operating range corresponds to the skin surface temperature between approximately 28° C. and 34° C.;
6) measuring the skin surface temperature at the first treatment location at least once during the treatment protocol;
7) adjusting the safe operating range for the parameters of the light source at the first treatment location, maintaining the skin surface temperature below the known pain threshold while simultaneously increasing the peak temperature and depth of the thermal gradient until at the correct depth, wherein the estimating, defining, measuring, and adjusting are updated continuously during the treatment; and
8) administering defining at least one higher-level laser pulse from the light source above the known pain threshold and below the damage threshold to raise a temperature of the chromophore to its required damage temperature.

3. The method of claim 2, further comprising: repeating steps 1) through 8) at a second treatment location on the first subject prior to administering the treatment protocol at the second treatment location.

4. The method of claim 2, further comprising: repeating steps 1) through 8) at the first treatment location on a second subject prior to administering the treatment protocol on the second subject.

5. The method of claim 2, further comprising:
9) storing in a memory of the photo-thermal targeted treatment system the safe operating range for the parameters for operating the light source for the first subject at the first treatment location; and
10) when administering the treatment protocol on the first subject at a later time, taking into consideration the parameters so stored in the memory.

6. The method of claim 2, further comprising:
9) if the skin surface temperature at the first treatment location reaches a preset threshold temperature, adjusting the parameters of the light source to reduce an effective power incident at the first treatment location.

7. The method of claim 2, wherein defining the safe operating range for the parameters of the light source includes setting at least one of laser power, pulse width, pulse interval, maximum power output, and a skin surface cooling mechanism.

8. The method of claim 2, further comprising: repeating steps 1) through 8) at a second treatment location on the first subject during administration of the treatment protocol at the second treatment location.

9. The method of claim 2, further comprising: repeating steps 1) through 8) at a first treatment location on a second subject during administration of the treatment protocol on the second subject.

10. The method of claim 9, further comprising:
9) storing in a memory of the parameters for operating the light source for the first subject at the first treatment location for the second subject; and
10) when administering the treatment protocol on the second subject at a later time, taking into consideration the parameters for operating the light source so stored in the memory.

11. A method for determining a suitable set of parameters for operating a light source within a photo-thermal targeted treatment system for targeting a chromophore embedded in a medium, the method comprising:
a) cooling a first treatment location;
b) administering at least one laser pulse from the light source at a preset power level to the first treatment location, the first laser pulse having thermal energy below a known pain and damage threshold of the medium;
c) tracking skin surface temperatures at the first treatment location at a refresh rate of 25 Hz to 400 Hz while administering the first laser pulse;
d) estimating a relationship between the parameters for operating the light source, post-pulse cooling and the skin surface temperature at the first treatment location by fitting the skin surface temperature and parameters for operating the light source using data correlations, wherein the predictor parameters are established using computational analysis taking into account a-priori knowledge of the correlation from clinical experiments;
e) defining an operating range for the parameters for operating the light source in order to stay below the pain threshold to the medium at the first treatment location while still effectively targeting the chromophore in administering the treatment protocol, wherein the safe operating range corresponds to the skin surface temperature between approximately 28° ° C. and 34° C.;
f) continuing to track skin surface temperatures at the first treatment location at a refresh rate of 25 Hz to 400 Hz while administering additional laser pulses;
g) adjusting the operating range for the parameters of the light source at the first treatment location, maintaining the skin surface temperature below a known pain threshold while simultaneously increasing a peak temperature and depth of the thermal gradient at the first treatment location until at a desired depth, wherein estimating, defining, measuring, and adjusting are updated continuously during a treatment protocol; and
h) defining at least one higher-level laser pulse from the light source above the known pain threshold and below the damage threshold to raise a temperature of the chromophore to its required damage temperature.

12. The method of claim 11, further comprising repeating steps a)-h) at a second treatment location on the first subject.

13. The method of claim 11, further comprising repeating steps a)-h) on a second subject.

14. The method of claim 11, wherein adjusting the parameters for operating the light source includes adjusting at least one of laser power, pulse width, pulse interval, maximum power output, and a skin surface cooling mechanism used for performing the cooling.

15. A method for treating a subject with a photo-thermal targeted treatment system including a light source for targeting a chromophore embedded in a medium, the method comprising:
a) cooling a first treatment location of the subject from a first surface temperature to a second surface temperature;
b) administering a laser pulse from the light source to the first treatment location;
c) during application of the laser pulse, tracking skin surface temperatures at the first treatment location at using an infrared camera operating at a refresh rate of 25 Hz to 400 Hz; and
d) terminating the treatment protocol based at least in part on the skin surface temperatures so measured.

16. The method of claim 15, wherein cooling the first treatment location includes cooling the first treatment location from the first surface temperature of body temperature to a second surface temperature less than body temperature.

17. The method of claim 15, wherein cooling includes using at least one of contact cooling, cooling air flow, and cryogen spray.

18. The method of claim 15, wherein tracking skin surface temperatures includes determining the skin surface temperatures at a refresh rate of at least 400 Hz.

19. The method of claim 15,
wherein terminating the treatment protocol includes fitting the skin surface temperatures so tracked to data correlations,
wherein the predictor parameters are established using computational analysis taking into account a-priori knowledge of the correlation from clinical experiments, determining appropriate laser parameters for the light source, and
the method further comprising:
modifying the treatment protocol according to the appropriate laser parameters.

20. The method of claim 19, wherein terminating the treatment protocol further includes determining appropriate cooling parameters for the cooling mechanism, and modifying the cooling parameters during the treatment protocol.

21. The method of claim 17,
wherein terminating a treatment protocol includes fitting the skin surface temperatures so tracked to data correlations,
wherein the predictor parameters are established using computational analysis taking into account a-priori knowledge of the correlation from clinical experiments,
the method further comprising:
predicting a peak skin surface temperature, and
adjusting at least one of laser power, pulse width, number of pulses, and cooling system parameters according to the peak skin surface temperature so predicted.

22. The method of claim 21, wherein the peak skin surface temperature is a temperature in a range of 40° C. and 55° C.

23. The method of claim 22, wherein the peak skin surface temperature is 45° C.

24. The method of claim 15, wherein a pulse duration of the laser pulse is 100 milliseconds.

25. The method of claim 15, wherein terminating the treatment protocol includes fitting the skin surface temperatures so tracked to data correlations, wherein the predictor parameters are established using computational analysis taking into account a-priori knowledge of the correlation from clinical experiments, determining appropriate laser parameters for the light source, and modifying the treatment protocol according to the appropriate laser parameters.

26. The method of claim 25, wherein terminating the treatment protocol further includes
determining appropriate cooling parameters for the cooling mechanism, and
modifying the cooling parameters during the treatment protocol.

27. The method of claim 26,
wherein terminating the treatment protocol includes fitting the skin surface temperatures so tracked to data correlations,
wherein the predictor parameters are established using computational analysis including
taking into account a-priori knowledge of the correlation from clinical experiments,
predicting a peak skin surface temperature, and
adjusting at least one of laser power, pulse width, number of pulses, and cooling system parameters.

28. The method of claim 27, wherein the peak skin surface temperature is a temperature in a range of 40° C. and 55° C.

29. The method of claim 28, wherein the peak skin surface temperature is in a range of 41° C. and 45° C.

30. The method of claim 15, wherein a pulse duration of the laser pulse is 100 milliseconds.

31. A photo-thermal targeted treatment system for targeting a chromophore embedded in a medium, the system comprising:
a cooling unit for providing cooling at a treatment location;
a light source for providing laser pulses at the treatment location;
a temperature monitoring unit for monitoring a skin surface temperature at the location; and
a controller for receiving the skin surface temperature as monitored by the temperature monitoring unit and accordingly controlling operating parameters of the cooling unit and the light source,
wherein the controller is configured for:
directing the light source to administer at least one laser pulse at a preset power to the treatment location, the preset power level being blow a known pain and damage threshold,
directing the temperature monitoring unit to measure a skin surface temperature at the treatment location,
correlation fitting a relationship between the operating parameters of the light source and the skin surface temperature so measured,
defining a safe operating range for the operating parameters of the light source in order to avoid pain and thermal damage to the medium at the treatment location,
modifying the operating parameters of at least one of the cooling unit and the light source to administer at least one higher-level laser pulse from the light source to maintain the skin surface temperature below the known pain and damage threshold while simultaneously increasing a peak temperature and depth of a thermal gradient until the peak temperature and depth of the thermal gradient reaches a desired depth within the medium at the treatment location, and
directing the light source to administer at least one treatment laser pulse from the light source with a power above the at least one initial laser pulse to raise a temperature of the targeted chromophore to its required damage temperature.
